(19) United States
(12) Patent Application Publication
(10) Pub. No.: US 2021/0087530 A1
(43) Pub. Date: Mar. 25, 2021

(54) COMPOSITIONS AND METHODS FOR CULTURING AND EXPANDING CELLS

(71) Applicant: LIFE TECHNOLOGIES CORPORATION, Carlsbad, CA (US)

(72) Inventors: Evan ZYNDA, Buffalo, NY (US); Sarya MANSOUR, Grand Island, NY (US); Anson PIERCE, Amherst, NY (US); Pei-Yi LIN, Amherst, NY (US); Nisha KAMATH, Cleveland Heights, OH (US)

(21) Appl. No.: 17/053,148

(22) PCT Filed: May 8, 2019
(86) PCT No.: PCT/US2019/031251

§ 371 Date: Nov. 5, 2020

Related U.S. Application Data
(60) Provisional application No. 62/668,369, filed on May 8, 2018.

(51) Int. Cl. C12N 5/0783

(52) U.S. Cl. CPC C12N 5/0636; C12N 2501/998; C12N 2500/02; C12N 2501/51; C12N 2501/2302; C12N 2501/515; C12N 2500/32

(57) ABSTRACT

Provided herein are improvements in mammalian cell culture. In partial particular, compositions, methods, and kits for culturing and expanding mammalian cells (e.g., immune cells, such as T cells and NK cells). In some aspects, compositions and methods are provided for enhancing the proliferation of cells in serum free media.

### RELATED APPLICATIONS

This application claims priority to and the benefit of U.S. Provisional Application No. 62/668,369 filed on May 8, 2018.

### FIELD OF THE INVENTION

Provided herein are improvements in mammalian cell culture. In partial particular, compositions, methods, and kits for culturing and expanding mammalian cells (e g, immune cells, such as T cells and NK cells). In some aspects, compositions and methods are provided for enhancing the proliferation of cells in serum free media.

### BACKGROUND

Mammalian cell cultivation presents a series of problems, especially for cell cultivation where the resulting cells are intended to be used for therapeutic purposes or research leading to potential therapeutic use.

When cells are to be used for therapeutic purposes, it is generally desirable to culture these cells in the absence of blood serum. Reasons for this include the possibility that the cells will become contaminated with adventitious agents present in serum (e.g., viruses, prions, Mycoplasma, etc.). Also, even sera pooled from the blood of a substantial number (e.g., 100 or more) animals has substantial lot to lot variability when used in mammalian cell culture (see FIG. 1). As can be seen from the data presented in FIG. 1, different lots of commercially available human serum show substantial variation in total T cell yields. Substantial variation as also found with respect to transduction efficiency (data not shown).

Additional issues with mammalian cell culture are low levels of viability, low cell density and, when performed, low levels of transduction efficiency.

Provided herein are improvements in mammalian cell culture. In partial particular aspects, compositions, methods, and kits for culturing and expanding mammalian cells (e.g., T cells) are provided. In some aspects, compositions and methods are provided for enhancing the proliferation of cells in serum free media.

### BRIEF SUMMARY

Compositions and methods are provided for the enhanced expansion of immune cells, such as immune cells, T cells, B cells and antigen presenting cells (APCs). Such compositions and methods provide conditions for favorable expansion of NK cells, T cells, B cells, and/or APCs. In some instances, compositions and methods provide suitable oxygen concentrations and/or lipid mixture for support rapid NK cell, T cell, B cell, and/or APC expansion.

In some instances, compositions (e.g., culture media, such as serum free culture media) and methods are provided for the expansion of mixed T cell populations where more than one T cell subtype expand at a similar rate. In many instances, T cell expansion will occur in the presence of serum albumin (e g, human serum albumin, such as recombinantly produced human serum albumin). Further, compositions and methods are provided for the expansion of T cell present in a mixed populations of T cells which allows for the depletion or enhancement of one or more T cell subtypes over one or more different T cell subtypes.

Also, provided herein are cell culture compositions and methods that allow for the cultivation of mammalian cells at high cell density and with high levels of cell viability.

Provided herein are compositions and methods for culturing, also referred to herein as expanding, immune cells (e.g., individual T cell subtypes or a mixed population of different T cell subtypes). In some instances, such methods comprise culturing the immune cells (e.g., NK cells, T cells, B cells, and/or APCs) under conditions where the immune cells (e.g., NK cells, T cells, B cells, and/or APCs) have a peak population maximum doubling time of from about 25 hours to about 40 hours (e.g., from about 25 hours to about 35 hours, from about 25 hours to about 32 hours, from about 25 hours to about 30 hours, from about 27 hours to about 35 hours, from about 30 hours to about 35 hours, from about 28 hours to about 40 hours, from about 30 hours, to about 40 hours, from about 29 hours, to about 39 hours, from about 28 hours, to about 37 hours, etc.) and wherein the T cells are cultured without serum.

Further, immune cells (e.g., NK cells, T cells, B cells, and/or APCs) may be cultured in the presence of serum albumin (e.g., human serum albumin) and this serum albumin may be produced recombinantly. When recombinant serum albumin is used, it may be produced either in a mammalian cell or non-mammalian cells (e.g., a yeast such a Pichia pastoris, or a plant, such as rice). The concentration of serum albumin in culture media employed in methods set out herein will typically be in the range of 0.1% to 1% (e.g., from about 0.1% to about 0.9%, from about 0.2% to about 0.9%, from about 0.3% to about 0.9%, from about 0.1% to about 0.8%, from about 0.1% to about 0.6%, from about 0.2% to about 0.5%, etc.).

In some methods, cells are cultured in a culture medium comprising OPTMIZER™ CTS™ SFM (Thermo Fisher Scientific, cat. no. A1048501) or in a culture medium comprising CTS™ Immune Cell Serum Replacement (ICSR) (Thermo Fisher Scientific, catalog number A2596101). In some methods, cells are cultured in a culture medium comprising OPTMIZER™ CTS™ SFM (Thermo Fisher Scientific, cat. no. A1048501) and CTS™ Immune Cell Serum Replacement (ICSR) (Thermo Fisher Scientific, catalog number A2596101).

Some aspects of compositions and methods set out herein relate to access of cell being cultured to oxygen and the removal of carbon dioxide. It is generally desirable for these cells to have ready access to oxygen with the efficient removal of carbon dioxide. Along these lines, typically cells cultured as set out herein will be present in an incubator. The O₂ concentration in such incubators will typically be between 15% and 25% (e.g., from about 15% to about 24%, from about 17% to about 25%, from about 18% to about 25%, from about 20% to about 25%, from about 22% to about 25%, from about 23% to about 25%, etc.). Further, the CO₂ concentration in such incubators will typically be between 2% and 7%.

In many instances, gas exchange will be facilitated by the use of a gas permeable membrane in contact with the culture media. Such membranes may be located at the bottom of a culture vessel and allow both for O₂ to enter culture media and for CO₂ to leave the culture media. In some instances, gas permeable membrane used in the practice of methods made be composed of or comprise gas permeable silicone and/or may be between 0.001 and 0.01 (e.g., from about 0.005 to about 0.007, from about 0.002 to about 0.007, from about 0.003 to about 0.007, from about 0.005 to about 0.009, from about 0.004 to about 0.008, etc.) inches in thickness.

In some methods, cells are cultured in a G-REX® culture vessel. Such as, for example, a G-REX® culture vessel selected from the group consisting of:

(A) a G-REX® 6M Well Plate (Wolf Wilson Corporation, part number 80660M),

(B) a G-REX® 24 Well Plate (Wolf Wilson Corporation, part number 80192M),

(C) a G-REX6® Well Plate (Wolf Wilson Corporation, part number 80240M),

(D) a G-REX® 10 (Wolf Wilson Corporation, part number 80040S),

(E) a G-REX100® 6M Well Plate (Wolf Wilson Corporation, part number 80500),

(F) a G-REX® 100M Well Plate (Wolf Wilson Corporation, part number 81100),

(G) a G-REX® 100M-CS Well Plate (Wolf Wilson Corporation, part number 81100-CS),

(H) a G-REX® 500M Well Plate (Wolf Wilson Corporation, part number 85500S), and

(I) a G-REX® 500M-CS Well Plate (Wolf Wilson Corporation, part number 85500-CS).

In some instances, it may be desirable for a glutamine source to be present in the media. When this is the case, then the glutamine source may be one that will not form substantial amounts of ammonia. One example of such a glutamine source is an L-alanyl-L-glutamine dipeptide. When present, such a glutamine reagent may be present at a concentration of between from about 1 mM to about 20 mM (e.g., from about 2 mM to about 20 mM, from about 5 mM to about 18 mM, from about 10 mM to about 20 mM, from about 8 mM to about 27 mM, etc.).

Further, while incubation temperatures for the cultivation of immune cells (e.g., NK cells, T cells, B cells, and/or APCs) may vary, immune cells (e.g., T cells) will typically be cultured at temperatures between 34° C. and 40° C.

Methods provided herein allow for the serum free cultivation of immune cells (e.g., NK cells, T cells, B cells, and/or APCs) with a high maximum doubling time, in the absence of serum, wherein the immune cells (e.g., NK cells, T cells, B cells, APCs, etc.) reach a population density of between 1.0×10⁷ and 8.0×10⁷ (e.g., from about 1.0×10⁷ to about 8.0×10⁷, from about 2.0×10⁷ to about 8.0×10⁷, from about 3.0×10⁷ to about 8.0×10⁷, from about 4.0×10⁷ to about 8.0×10⁷, from about 3.0×10⁷ to about 6.5×10⁷, etc.) cells per cm².

Immune cells (e.g., NK cells, T cells, B cells, APCs, etc.) used in compositions and methods set out herein may be obtained from sample provided by donors (e.g., human donors, mouse donors, etc.).

Further, immune cells (e.g., NK cells, T cells, B cells, APCs, etc.) used in compositions and methods set out may be contacted with one or more agents (e.g., one or more antibodies) that bind to one or more cell surface receptors. Such agents may be used to purify a subset of, for example, T cells from other T cells or to separate T cells from non-T cells (e.g., such as NK cells, B cells, APCs, etc.). Such agents may be also used to “activate” some or all of the T cells present (e.g., one or more T cell subtype). Such agents one or more agents comprise one or more antibody or antibody fragment (or other agent) that binds to one or more T cell surface receptor selected from the group consisting of: (a) CD3 receptors, (b) CD4 receptors, (c) CD5 receptors, (d) CD6 receptors, (e) CD28 receptors, (f) CD137 receptors, and/or (g) CD278 receptors.

Also provided herein are compositions and method for preferentially expanding one or more subsets of T cell present in a mixed population of T cells. In some instances, immune cells (e.g., NK cells, T cells, B cells, and/or APCs) may be expanded in the absence of serum and/or the immune cells may be expanded in a culture vessel having a gas permeable membrane. In some methods, expanding immune cells (e.g., NK cells, T cells, B cells, and/or APCs) may have a maximum doubling time of from about 25 hours to about 40 hours. Further, T cells may be expanded in the presence of one or more chemokine or cytokine (e.g., one or more chemokine or cytokine selected from the group consisting of: (a) Interleukin-1α, (b) Interleukin-2, (c) Interleukin-4, (d) Interleukin-1β, (e) Interleukin-6, (f) Interleukin-12, (g) Interleukin-15, (h) Interleukin-18, (i) Interleukin-21, and/or (j) Transforming growth factor β1.

Further provided are compositions and their use in methods where one or more T cell subset preferentially expands over one or more different T cell subset. As an example, in some aspects provided herein, memory T cells may preferential expand over antigen specific T cells.

Further provided are compositions and their use in methods where the total T cell population expands at a rate that is from 5 to 15 times faster than antigen specific T cells. As used here, the phrase “total T cell population” refers to a mixed population of T cells, such as a mixed population obtained from a donor.

Further provided are compositions and their use in methods where memory T cells expand at a rate that is from 5 to 15 times faster than antigen specific T cells and where regulatory T cells expand at a rate that is from 5 to 15 times faster than antigen specific T cells.

Also provided herein are compositions and methods for the activation and expansion of T cells. In some instances such methods may comprise: (a) activating the T cells, and (b) expanding the T cells, wherein the T cells are expanded under conditions wherein they have a maximum doubling time of from about 25 hours to about 40 hours, and wherein the T cells are expanded in the absence of serum. In some instances, the T cells may be purified prior to activation. Further, this purification may be by negative selection or positive selection. While numerous methods may be employed for selection, negative selection or positive selection may occur by either removing or collecting T cells by the use of one or more agents that bind to CD2 receptors or CD3 receptors. Such one or more agents that bind to CD2 receptors or CD3 receptors may be anti-CD2 and anti-CD3 antibodies.

Further provided are compositions and their use in methods for expanding cells of one or more T cell subsets. Such methods may comprise: (a) purifying members of a T cell subset, (b) culturing the members of the T cell subset obtained in (a), wherein the T cells are expanded under conditions wherein they have a maximum doubling time of from about 25 hours to about 40 hours, and wherein the T cells are expanded in the absence of serum. Such T cell subsets may be selected from the groups consisting of: (a) Th1 T cells, (b) Th2 T cells, (c) Th17 T cells, (d) Th22 T cells, (e) regulatory T cells, (f) naïve T cells, (g) antigen specific T cells, (h) central memory T cells, (i) effector memory T cells, (j) tissue resident memory T cells, and (k) virtual memory T cells. Further, members of the T cell subset may be purified by (1) selective expansion and/or (2) positive of negative selection. Further, negative selection or positive selection occur by either removing or collecting T cells by the use of one or more agents that bind to one or more cell surface markers, such as one or more cell surface marker surface marker selected from the group consisting of: (a) CD2 receptors, (b) CD3 receptors, (c) CD4 receptors, (d) CD8 receptors, (e) CD19 receptors, (f) CD20 receptors, and/or (g) CD28 receptors. Further, the one or more agents that bind to the one or more surface markers may be anti-surface marker antibodies.

Further provided are compositions and their use in methods for generating a population of activated, engineered T the immune cells (e.g., NK cells, T cells, B cells, APCs, etc.). Such methods may comprise: (a) introducing into the population of the immune cells (e.g., NK cells, T cells, B cells, APCs, etc.) a nucleic acid molecule that encodes protein (e.g., a fusion protein) under conditions where the protein is expressed in the immune cells (e.g., NK cells, T cells, B cells, APCs, etc.), wherein the protein is a cell surface protein, to produce a population of engineered the immune cells (e.g., NK cells, T cells, B cells, APCs, etc.), (b) activating members of the population of engineered the immune cells, and (c) expanding activating members of the population of engineered immune cells to produce the population of activated, engineered immune cells, wherein the immune cells are expanded under conditions wherein they have a maximum doubling time of from about 25 hours to about 40 hours, and wherein the immune cells are expanded in the absence of serum. Also provided are methods further comprising purifying one or more T cell subset prior to introducing into the population of T cells the nucleic acid molecule that encodes protein. In many instances, the nucleic acid molecule that encodes protein will be introduced into the one or more T cell subset that is purified. Protein encoded by nucleic acid molecules introduced into the one or more T cells will often be fusion protein (e.g., one or more chimeric antigen receptors). Further, T cell subsets (e.g., engineered T cell subset) and populations of T cells (e.g., populations of engineered T cells) expanded as set out herein may be expanded in the presence of one or more one cytokine (e.g., Intereukin-2).

### BRIEF DESCRIPTION OF THE DRAWINGS

FIG. 1. Human serum shows inconsistency between lots. Human T cells were expanded in basal media supplemented with several unqualified lots of human serum (huAB) compared to a control lot of human serum. OPTMIZER™ medium with 5% human serum. Growth was measured over a 10 day course following stimulation.

FIG. 2. T cell expansion in the G-REX® culture vessels (G-REX® 6M Well Plate, cat. no. 80660M) using various serum-free basal media. T cells were cultured in the indicated serum-free basal media for 10 days. (A) Cell growth was measured over time and reported in fold expansions. (B) Phenotypic characterization was performed on day 10 to determine the CD8:CD4 ratio and (C) the degree of differentiation, both of which are reported relative to the percent of the CD3+ cell population. These experiments employed X-VIVO-15™ (Lonza, cat. no. BE02-060Q) containing 5% hABs (human serum) as a benchmark. Data is representative of three replicates. Each block of three columns in (C) is, from left to right, X-VIVO-15™ with hABS, X-VIVO-15™, and OPTMIZER™.

FIG. 3. CTS™ Immune Cell Serum Replacement (ICSR) (Thermo Fisher Scientific, cat. no. A2596101) enhances T cell growth using serum-free media in the G-REX® culture vessel (cat. no. 80660M). T cells were cultured in the indicated serum-free media supplemented with 2.5% CTS™ Immune Cell Serum Replacement (ICSR) for 10 days. Cell growth was measured over time and reported in fold expansions. Each panel (A-C) displays the data for primary T cells isolated from independent donors. These investigations employed X-VIVO-15™ containing 5% hABs (i.e., human serum) as a benchmark.

FIG. 4. T cells expanded in serum-free medium containing serum replacement exhibit memory phenotype. T cells were cultured in the indicated serum-free media supplemented with 2.5% CTS™ Immune Cell Serum Replacement (ICSR) for 10 days. Phenotypic characterization was performed on day 10 to determine the CD8:CD4 ratio (Panels A, C, and E) and the degree of differentiation (Panels B, D, and F), both of which are reported relative to the CD3+ cell population. Each panel (Panels A-F) displays the data for primary T cells isolated from three independent donors. These investigations employed X-VIVO-15™ containing 5% human serum (hABs) as a benchmark. Each block of four columns in Panels A, C, and E is, from left to right, X-VIVO-15™ with hABS, X-VIVO-15™ ICSR, AIM-V ICSR, and OPTMIZER™ ICSR.

FIG. 5. A comparison of expansions fold of T cells expanded in for 10 days in OPTMIZER™ CTS™ SFM supplemented with CTS™ Immune Cell Serum Replacement (ICSR), where the T cells were expanded in either static plates or in the G-REX® system. These data were generated as set out in Example 2.

FIG. 6. A comparison of the CD4/CD8 ratios of cell expanded as set out in the legend for FIG. 5.

FIG. 7. A comparison of expansions fold of T cells expanded in for 10 days in OPTMIZER™ CTS™ SFM supplemented with CTS™ Immune Cell Serum Replacement (ICSR), where the T cells were expanded in either the XURI™ system or in the G-REX® system. These data were generated as set out in Example 2.

FIG. 8. A comparison of the CD4/CD8 ratios of cell expanded as set out in the legend for FIG. 7.

FIG. 9. An exemplary cell culture vessel containing a gas permeable membrane.

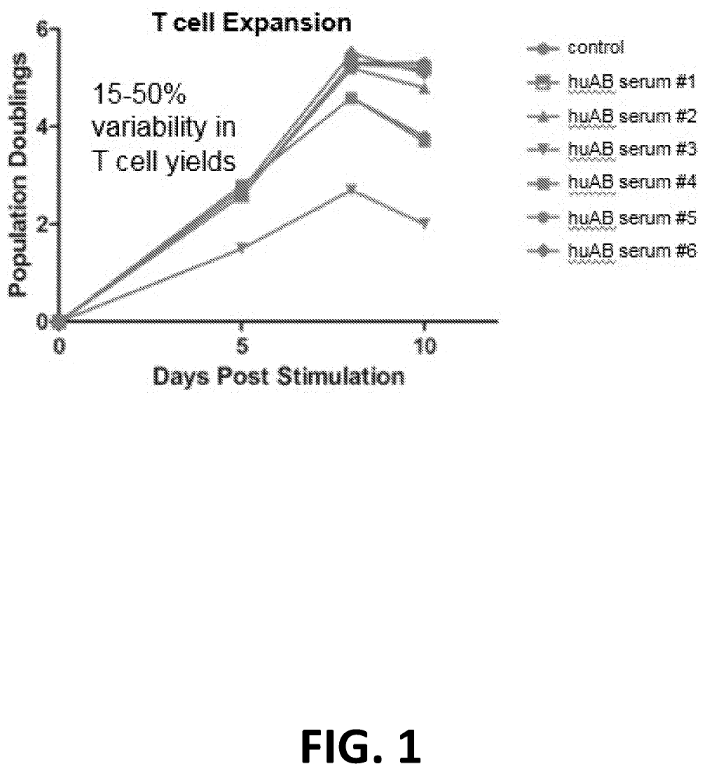

### DETAILED DESCRIPTION

### Definitions

The following definitions are included for the purpose of understanding the present subject matter and for constructing the appended patent claims. Abbreviations used herein have their conventional meaning within the chemical and biological arts.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention.

As used herein, the term “about” in the context of a numerical value or range means ±10% of the numerical value or range recited or claimed, unless the context requires a more limited range.

As used herein, the term “culture medium” or “culture media” refers to a composition that provides for the growth and/or viability of cells. Culture media may be in dried a formal or may be liquid (e.g., an aqueous solution). Further, culture media may be chemically defined in that they are prepared by the combining of specific compounds. Chemically defined media may also be supplemented with additional agent such as sera. Examples of culture media include Dulbecco's Modified Eagle Medium (DMEM), Iscove's Modified Dulbecco's Medium, Minimum Essential Medium (MEM), Ham's F-10, Ham's F-12, and Roswell Park Memorial Institute 1640 Medium (RPMI).

As used herein “serum replacement” or “serum replacement media” refers to a composition that can be used in conjunction with culture media to promote cell growth and survival of cells (e.g., T cells). In various embodiments, serum replacement may contain compounds such as salts, amino acids, vitamins, trace elements, antioxidants, and proteins (e.g., cytokines, chemokines, recombinant serum albumin, etc.).

As used herein, “culture medium supplement” refers to an agent or composition that may be added to culture media to allow for or enhance the growth and/or viability of cells. Culture medium supplements may contain growth factors, hormones, proteins, serum or serum replacement, trace elements, sugars, antibiotics, antioxidants, etc.

As used herein, the term “gas permeable membrane” is a layer that allows gas to pass through. Gases these membranes may be permeable to include O₂, CO₂, and N₂. Gas permeable silicone membranes approximately 0.005 to 0.007 inches thick may be used and are referred to U.S. Pat. No. 9,567,565. Further, cell culture devices containing such gas permeable membranes include those in the G-REX® series, which may be obtained from Wilson Wolf Corporation, 33 5th Ave NW, Saint Paul, Minn. 55112 (see, e.g., P/Ns 85500S-CS and 81100S). Other examples of gas permeable membranes and devices containing them are gas permeable pates available from Coy Lab Products (see cat. no. 8602000). These plates allow for the control of O₂ levels that cells are contacted with in incubators from your incubators. Specifications of these plates are as follows: 25 μm polymer film which allows high gas transfer rate while retaining liquid, O₂ permeability greater than 9000 cm³/M², CO₂ permeability greater than 7000 cm³/M².

As used herein, the term “atmospheric oxygen level” refers to an oxygen level that is about 20.95%.

As used herein, the term “doubling time”, with respect to cell replication refers to the amount of time it takes for a population of cells to double in number. For example, if a at one time point, a population of cells is composed of 100,000 cells and the cell population replicates to form 200,000 cells, then the time period between the time that there are 100,000 cells and when there are 200,000 cells is the doubling time. The doubling time is generally based upon the number of viable cells at the earlier time point. Further, doubling time may be linear or include a may reflect increasing and decreasing cell division rates at different time points in the replication process.

As used herein, the term “maximum doubling time”, refers to the point in the growth phase where the doubling time is the most rapid. In many instances, the cells in the population will begin with a lower doubling time when put under conditions designed to enhance cell expansion. Typically, after one or two cell divisions, the cells will typically have fully adjusted to the favorable conditions and the doubling time will increase and reach what is referred to as the maximum doubling time. This enhanced growth rate will generally continue until waste products accumulate or nutrients are depleted.

The term “activation,” as used herein, refers to the state of a cell following sufficient cell surface moiety ligation to induce a measurable morphological, phenotypic, and/or functional change. Within the context of T cells, such activation may be the state of a T cell that has been sufficiently stimulated to induce cellular proliferation. Activation of a T cell may also induce cytokine production and/or secretion, and up- or down-regulation of expression of cell surface molecules such as receptors or adhesion molecules, or up- or down-regulation of secretion of certain molecules, and performance of regulatory or cytolytic effector functions. Within the context of other cells, this term infers either up- or down-regulation of a particular physico-chemical process.

In embodiments, stimulation comprises a primary response induced by ligation of a cell surface moiety. For example, in the context of receptors, such stimulation may entail the ligation of a receptor and a subsequent signal transduction event. In embodiments, culturing T cells comprises stimulating the T cells. With respect to stimulation of a T cell, such stimulation may refer to the ligation of a T cell surface moiety that in embodiments subsequently induces a signal transduction event, such as binding the TCR/CD3 complex. In embodiments, the stimulation event may activate a cell and up- or down-regulate expression of cell surface molecules such as receptors or adhesion molecules, or up- or down-regulate secretion of a molecule, such as down-regulation of Tumor Growth Factor beta (TGF-β) or up-regulation of IL-2, IFN-γ etc. In embodiments, ligation of cell surface moieties, even in the absence of a direct signal transduction event, may result in the reorganization of cytoskeletal structures, or in the coalescing of cell surface moieties, each of which could serve to enhance, modify, or alter subsequent cell responses.

The term “ligand” or “stimulatory agent”, as used herein, refers to a molecule that binds to one or more defined population of cells (e.g., members of T cell subpopulations) and induces a cellular response. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), or the like. In embodiments, in the context of T cell stimulation, antibodies are used as a prototypical example of such an agent.

Antibodies for use in methods set out herein may be of any species, class or subtype providing that such antibodies can react with the target of interest, e.g., CD3, the TCR, or CD28, as appropriate.

Thus “antibodies” for use in methods set out herein include:

(a) any of the various classes or sub-classes of immunoglobulin (e.g., IgG, IgA, IgM, IgD or IgE derived from any animal, e.g., any of the animals conventionally used, e.g., sheep, rabbits, goats, mice, camelids, or egg yolk),

(b) monoclonal or polyclonal antibodies,

(c) intact antibodies or fragments of antibodies, monoclonal or polyclonal, the fragments being those which contain the binding region of the antibody, e.g., fragments devoid of the Fc portion (e.g., Fab, Fab′, F(ab′)2, scFv, V_{H}H, or other single domain antibodies), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains.

(d) antibodies produced or modified by recombinant DNA or other synthetic techniques, including monoclonal antibodies, fragments of antibodies, “humanized antibodies”, chimeric antibodies, or synthetically made or altered antibody-like structures.

Also included are functional derivatives or “equivalents” of antibodies e.g., single chain antibodies, CDR-grafted antibodies etc. A single chain antibody (SCA) may be defined as a genetically engineered molecule containing the variable region of the light chain, the variable region of the heavy chain, linked by a suitable polypeptide linker as a fused single chain molecule.

The term “differentiation”, as used herein, refers to a stage in development of the life cycle of a cell. T cells originate from hematopoietic stem cells in the bone marrow and generate a large population of immature thymocytes. The thymocytes (or T cells) progress from double negative cells to become double-positive thymocytes (CD4+CD8+), and finally mature to single-positive (CD4+CD8- or CD4-CD8+). During T cell differentiation, the naïve T cell becomes a blast cell that proliferates by clonal expansion and differentiates into memory and effector T cells. Many subsets of helper T cells (Th cells) are created during T cell differentiation and perform different functions for the immune system. In some embodiments, the differentiation stage of a T cell may be assessed through the presence or absence of markers including, but not limited to, CD3, CD4, CD5, CD8, CD11c, CD14, CD19, CD20, CD25, CD27, CD33, CD34, CD45, CD45RA, CD45RB, CD56, CD62L, CD123, CD127, CD278, CD335, CD11a, CD45RO, CD57, CD58, CD69, CD95, CD103, CD161, CCR7, as well as the transcription factors FOXP3, RORγ, T-bet, c-Rel, GATA3, etc.

A “co-stimulatory signal,” as used herein, refers to a signal, which in combination with a primary signal, such as TCR/CD3 ligation, leads to T cell proliferation and/or activation and/or polarization.

“Separation,” as used herein, includes any means of substantially purifying one component from another (e.g., by filtration, affinity, buoyant density, or magnetic attraction).

As used, herein, the term “purifying” refers enhancing the amount of a component of a mixture over one or more other components. As an example, assume that Treg cells are in a mixed population of T cells where the Treg cells represent 5% of the populations and all of the other T cells represent 95% of the total T cell population. If a process is performed that renders 20% of the population Treg cells with the other T cells representing 80% of the total T cell population, the Treg cells have been “purified”. Typically, when a T cell subset has been purified, the ratio of the T cell subset will be increased by at least two fold (e.g., from a 1:10 ratio to a 1:5 ratio) (e.g., from about two fold to about 100 fold, from about two fold to about 100 fold, from about 2 fold to about 100 fold, from about 5 fold to about 100 fold, from about 8 fold to about 100 fold, from about 15 fold to about 100 fold, from about 10 fold to about 40 fold, etc.).

As used, herein, the terms “immune cells” and “immune system cells” refer to cells that are involved in immunological responses designed to protect organisms from foreign substances, viruses, and cells Immune cells may be derived from a number of organs and tissues, such as the thymus, spleen, lymph nodes, clusters of lymphoid tissue (as in the gastrointestinal tract and bone marrow). Such cells include T cells, B cells, natural killer cells, macrophages, neutrophils, tumor infiltrating lymphocytes, dendritic cells, mast cells, eosinophils, and basophils, as well as progenitor cells that develop into these cells.

As used, herein, the term “CD8+ T cell” refers to a T cell that presents the co-receptor CD8 on its surface. CD8 is a transmembrane glycoprotein that serves as a co-receptor for T cell receptor (TCR), which can recognize a specific antigen. Like the TCR, CD8 binds to a major histocompatibility complex I (MHC I) molecule. In embodiments, CD8+ T cells are cytotoxic CD8+ T cells (also known as cytotoxic T lymphocytes, T-killer cells, cytolytic T cells, or killer T cells). In embodiments, CD8+ T cells are regulatory CD8+ T cells, also referred to as CD8+ T cell suppressors.

As used, herein, the term “CD4+ T cell” refers to a T cell that presents the co-receptor CD4 on its surface. CD4 is a transmembrane glycoprotein that serves as a co-receptor for T cell receptor (TCR), which can recognize a specific antigen. In embodiments, CD4+ T cells are T helper cells. T helper cells (TH cells) assist other white blood cells in immunologic processes, including maturation of B cells into plasma cells and memory B cells, and activation of cytotoxic T cells and macrophages. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate different types of immune responses. Signaling from the APC directs T cells into particular subtypes. In embodiments, CD4+ T cells are regulatory T cells.

Included herein are methods for efficiently generating regulatory T cells (or “T regulatory cell” or “Treg”) and the use of these methods in the generation of T cell populations which have applications in, for example, immunotherapy. Treg cells can be characterized by markers, such as CD4+, CD25+, FOXP3+, CD127^{neg/low}. In embodiments, Treg cells expanded using compositions and methods provided herein are CD4+, CD25+, FOXP3-. Non-limiting examples of compositions and methods for generating FOXP3-regulatory T cells are set out in Aarvak et al., U.S. Pat. No. 9,119,807.

Without being bound by any scientific theory, naturally occurring regulatory T (Treg) cells negatively regulate the activation of other T cells, including effector T cells, as well as innate immune system cells and can be utilized in immunotherapy against autoimmune diseases and provide transplantation tolerance. Various populations of Treg cells have been described and include naturally occurring CD4+CD25+FOXP3+ cells and induced Tr1 and Th3 cells that secrete IL-10 and TGF-β, respectively.

Treg cells are characterized by sustained suppression of effector T cell responses. Traditional or conventional Treg cells can be found, e.g., in the spleen or the lymph node or in the circulation. Tregs are proven highly effective in preventing GVHD and autoimmunity in murine models. Clinical trials with adoptive transfer of Tregs in transplantation, treatment of diabetes and other indications are underway. The relative frequency of Tregs in peripheral blood is approximately 1-2% of total lymphocytes implicating the necessity of ex vivo expansion of Tregs prior to adoptive transfer for most clinical applications. Producing sufficient Tregs during the ex vivo expansion has been a major challenge in applying Treg therapy to humans.

T helper 17 cells (or “Th17 cells” or “Th17 helper cells”) are an inflammatory subset of CD4+T helper cells that regulate host defense, and are involved in tissue inflammation and various autoimmune diseases. Th17 cells have been found in various human tumors however their function in cancer immunity is unclear. When adoptively transferred into tumor-bearing mice, Th17 cells have been found to be more potent at eradicating melanoma than Th1 or non-polarized (Th0) T cells (Muranski et al. “Tumor-specific Th17 polarized cells eradicate large established melanoma”, Blood 112:362-373 (2008)). Th17 cells are developmentally distinct from Th1 and Th2 lineages. Th17 cells are CD4+ cells that are responsive to IL-1R1 and IL-23R signaling and produce the cytokines IL-17A, IL-17F, IL-17AF, IL-21, IL-22, IL-26 (human), GM-CSF, MIP-3α, and TNFα. The phenotype of Th17 cells is controversial but currently defined as CD3⁺, CD4⁺, CCR4+, CCR6+ or CD3+, CD4+, CCR6+, CXCR3+. One obstacle to the use of Th17 cells for adoptive cell transfer has been the identification of robust culture conditions that can expand the Th17 cell subset.

Included herein are compositions and methods for the generation of T cell subtypes. One T cell subtype that may be produced using compositions and methods set out herein is Th17 cells.

T helper 9 cells (or “Th9 cells” or “Th9 helper cells”) are an inflammatory subset of CD4+T helper cells that regulate host defense and are involved in allergy, inflammation and various autoimmune diseases. Th9 cells are identified by secretion of the signature cytokine IL-9. Although Th9 cells share some functional roles with Th2 cells, including promoting allergic inflammation and helminthic parasite immunity, Th9 cells can also promote autoimmunity in responses that are generally characterized as dependent on Th1 or Th17 cells. Th9 cells are differentiated under a cytokine environment containing both IL-4 and transforming growth factor β (TGFβ), which induce the transcriptional network required for the expression of IL-9. The Th9 subset is defined by its ability to produce large amounts of the signature cytokine IL-9. Transcription factors required for the development of Th9 cells include signal transducer and activator of transcription-6 (STAT6), interferon regulatory factor 4 (IRF4), B-cell activating transcription factor-like (BATF), GATA3, PU.1 and Smads. Th9 cells express high levels of IL-25 receptor (IL17RB), which is a potential surface maker to distinguish Th9 cells from other T helper subsets Immune responses mediated by Th9 cells contribute to the protective immunity against intestinal parasite infection and to anti-tumor immunity.

Provided herein are compositions and methods for the generation of T cell subtypes. A non-limiting example of a T cell subtype that may be produced using compositions and methods set out herein are Th9 cells.

Memory T cells, or antigen-experienced cells, are experienced in a prior encounter with an antigen. These T cells are long-lived and can recognize antigens and quickly and strongly affect an immune response to an antigen to which they have been previously exposed. Memory T cells can encompass: stem memory cells (T_{SCM}), central memory cells (T_{CM}), effector memory cells (T_{EM}). T_{SCM} cells have the phenotype CD45RO−, CCR7+, CD45RA+, CD62L+(L-selectin), CD27+, CD28+ and IL-7Rα+, but they also express large amounts of IL-2Rβ, CXCR3, and LFA-1. T_{CM} cells express L-selectin and CCR7, and they secrete IL-2. T_{EM} cells do not express L-selectin or CCR7 but produce effector cytokines like IFN-γ and IL-4.

Included herein are methods and compositions for the expansion of T cell populations (e.g., mixed populations of T cells and T cell subsets).

“Chimeric antigen receptor” or “CAR” or “CARs” as used herein refers to engineered receptors, which graft an antigen specificity onto cells (for example T cells such as naive T cells, central memory T cells, effector memory T cells or any combination thereof). CARs are also known as artificial T cell receptors, chimeric T cell receptors or chimeric immunoreceptors. In embodiments, a CAR comprises one or more antigen-specific targeting domains, an extracellular domain, a transmembrane domain, one or more co-stimulatory domains, and an intracellular signaling domain. In embodiments, if the CAR targets two different antigens, the antigen-specific targeting domains may be arranged in tandem. In embodiments, if the CAR targets two different antigens, the antigen-specific targeting domains may be arranged in tandem and separated by linker sequences.

CARs are engineered receptors, which graft an arbitrary specificity onto an immune effector cell (T cell). These receptors are used to graft the specificity of a monoclonal antibody onto a T cell; with transfer of their coding sequence facilitated by retroviral vectors. The receptors are called chimeric because they are composed of parts from different sources. CARs may be used as a therapy for cancer through adoptive cell transfer. T cells are removed from a patient and modified so they express receptors specific to the patient's particular cancer. The T cells, which recognize and kill the cancer cells, are reintroduced into the patient. In embodiments, modification of T cells sourced from donors other than the patient may be used to treat the patient.

Using adoptive transfer of T cells expressing chimeric antigen receptors, CAR-modified T cells can be engineered to target any tumor-associated antigen. Following the collection of a patient's T cells, the cells are genetically engineered to express CARs specifically directed towards antigens on the patient's tumor cells before being infused back into the patient.

A method for engineering CAR T cells for cancer immunotherapy is to use viral vectors such as retrovirus, lentivirus or transposon, which integrate the transgene into the host cell genome. Alternatively, non-integrating vectors such as plasmids or mRNA may be used but these types of episomal DNA/RNA may be lost after repeated cell division. Consequently, the engineered CAR T cells may eventually lose their CAR expression. In another approach, a vector is used that is stably maintained in the T cell, without being integrated in its genome. This strategy has been found to enable long-term transgene expression without the risk of insertional mutagenesis or genotoxicity.

As used herein, the term “NK cells” refers to immune cells that are lymphocytes that mediate anti-tumor and anti-viral responses. NK cells do not express polymorphic clonotypic receptors and utilize inhibitory receptors (killer immunoglobulin-like receptor and Ly49) to develop, mature, and recognize “self” from “non-self.” Human natural killer (NK) cells can be divided generally into different groups based on the relative expression of the surface markers CD56 and CD16. The two major groups of these cells are CD56⁺, CD16^{Low/-} and CD56^{Low/-}, CD16⁺.

As used, herein, the term antigen presenting cells (APCs) refers to a group of immune cells that mediate the cellular immune response by processing and presenting antigens for recognition by certain lymphocytes such as T cells. Such cells APCs include dendritic cells, macrophages, Langerhans cells and B cells.

### Combinations and Methods of Culturing

Provided herein are compositions of different compounds, as well as methods for preparing and/or using such compositions. In particular aspects, provided herein are cell culture compositions and methods for the expansion of mammalian cells in the absence of serum. In some aspect, cell culture methods are provided that allow for the cultivation of mammalian cells (e.g., T cells) with a high maximum doubling time and to high cell density.

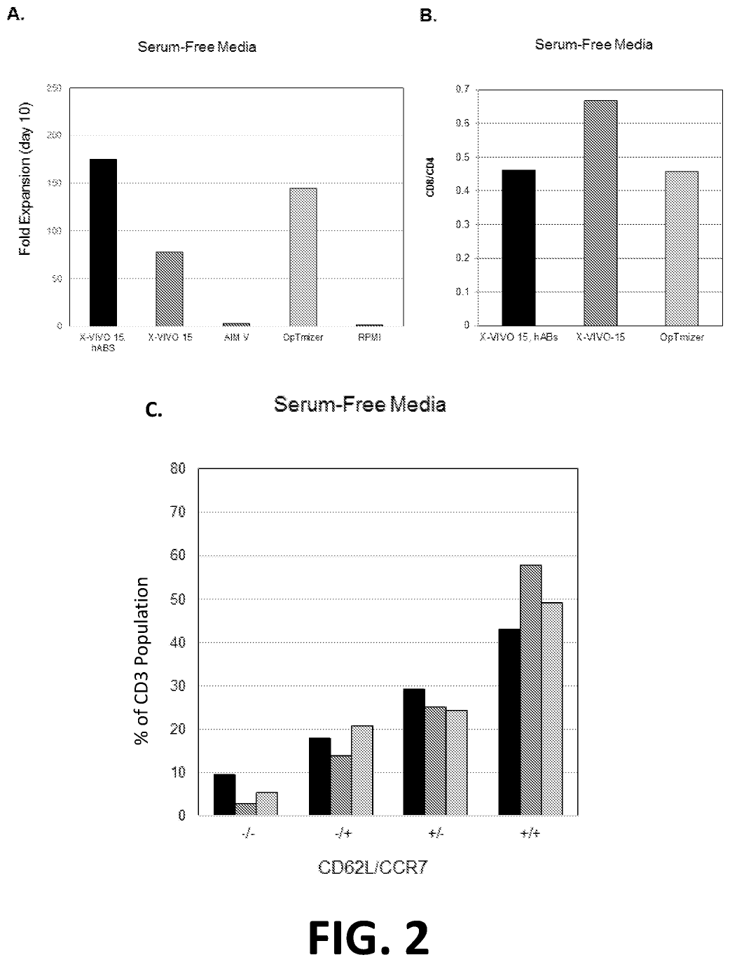

Exemplary data generated using compositions and methods set out herein are provided in FIG. 2A and in Table 1. Further, some of the data set out in Table 1 was used to prepare FIG. 2A.

TABLE 1
T cell expansion in the G-REX ® culture vessels
using various serum-free basal media (See FIG. 2)

| Days of | X-VIVO 15 ™, | | X-VIVO 15 ™, | | |
|---|---|---|---|---|---|
| Culture | hABS | X-VIVO 15 ™ | ICSR | AIM-V | AIM-V, ICSR |
| 0 | 0 | 0 | 0 | 0 | 0 |
| 7 | 72.21667 | 39.31083 | 61.25 | 1.575 | 28.11667 |
| 10 | 174.5333 | 78.05 | 119.35 | 2.8175 | 58.85833 |

| | OPTMIZER ™ | OPTMIZER ™, ICSR | RPMI | RPMI, ICSR |
|---|---|---|---|---|
| 0 | 0 | 0 | 0 | 0 |
| 7 | 64.925 | 81.08333 | 0.974167 | 1.230833 |
| 10 | 144.55 | 182.2917 | 0.583333 | 0.635833 |

It is believed that the high cells growth rate and expansion level provided by compositions and methods set out herein result, in part, from one or more of the following factors: (1) Efficient gas exchange between the culture media and surrounding environment, (2) the presence of a glutamine source may be one that will not form substantial amounts of ammonia, and (3) the presence of serum albumin.

With respect to gas exchange, gas permeable membranes may be employed which allow for O₂ and CO₂ exchange, do not allow for significant leakage of fluid from the culture and are impermeable to microorganism.

Typically, gas permeable membranes will be in direct contact with the culture media (e.g., will be at the bottom if the culture vessel) and will have a suitable surface area related to the volume of the culture media to allow for suitable gas exchange.

With respect to cylinders, the surface are is defined by the equation:

A=2πrh+2πr²,

where r is the radius and h is the height. If the r and h=1 unit, then the area is 12.57 units, with each end of the cylinder being 3.14 (π). If it is assumed that a gas permeable membrane is located only one end of the cylinder, then the ratio of the surface area of that end to the total surface area is approximately 1:4.

Compositions and methods set out herein will generally be directed to cell cultures where the gas permeable membrane to total surface area ratio will be in the range of 1:2.5 to 1:100 (e.g., from about 1:3 to about 1:100, from about 1:4 to about 1:100, from about 1:5 to about 1:100, from about 1:8 to about 1:100, from about 1:10 to about 1:100, from about 1:15 to about 1:100, from about 1:3 to about 1:90, from about 1:3 to about 1:75, from about 1:8 to about 1:75, from about 1:12 to about 1:50, etc.).

Further, as mammalian cell perform aerobic metabolic processes, these cells consume O₂ and generate CO₂ Culture conditions will typically be adjusted maintain O₂ and CO₂ at levels suitable to enhance cell expansion. With respect to O₂, this may be done by altering the O₂ level in the external local environment (e.g., the incubator).

Further, since gas exchange through gas permeable membranes is based upon diffusion, O₂ and CO₂ levels in the external local environment (e.g., the incubator) may be adjusted to arrive at desired in culture media. Thus, 02 levels may be maintained at between 15% and 25% (e.g., from about 15% to about 24%, from about 17% to about 25%, from about 18% to about 25%, from about 20% to about 25%, from about 22% to about 25%, from about 23% to about 25%, etc.). Further, CO₂ levels may be independently maintained at between 1% and 7% (e.g., from about 1% to about 6%, from about 1% to about 5%, from about 1% to about 3%, from about 2% to about 6%, from about 2% to about 4%, etc.).

As noted elsewhere herein, compositions and methods provided herein are directed, in part, to the serum free culture of mammalian cells (e.g., T cells) with a rapid maximum population doubling time, to high cells density, and with high cell viability.

The cell density of cultured cells is determined by a number of factors, including nutrient levels and waste product buildup. Put another way, the resources available to the cells and the presence of compounds that are inhibitory and/or toxic. In particular, compositions and methods are provided here that allow for the culturing of mammalian cells to cell densities in the range of 1.0×10⁷ and 8.0×10⁷ per cm².

Also, provide herein are compositions and methods that allow for the culturing of mammalian cells to cell densities in the range of 3.0×10⁶ to 5.0×10⁷ per cm³ (e.g., from about 3.0×10⁶ to 5.0×10⁷, from about 4.0×10⁶ to 5.0×10⁷, from about 5.0×10⁶ to 5.0×10⁷, from about 6.0×10⁶ to 5.0×10⁷, from about 7.0×10⁶ to 5.0×10⁷, from about 9.0×10⁶ to 5.0×10⁷, from about 9.0×10⁶ to 4.0×10⁷, etc. per cm³). In some instances, compositions and methods that allow for the culturing of mammalian cells to cell densities in the range 3.0×10⁶ to 5.0×10⁷ per cm³ wherein the cell viability is in the range of 80% to 100% (e.g., from about 82% to about 100%, from about 82% to about 100%, from about 84% to about 100%, from about 85% to about 100%, from about 80% to about 98%, etc.). As set out elsewhere herein, these cells may be expanded in the absence of serum and/or may have expanded at maximum doubling time of from about 25 hours to about 40 hours.

Also provided herein are compositions and methods that allow for the production of T cell populations with high levels of cell. While the percentage of viable cells in a cell culture various with a number of factors, including the stage of the growth phase (e.g., the duration of time before “lag” phase will occur), the cell type, and the availability of metabolic resources, compositions and methods provided herein allow for the production of cell populations where the cell viability is greater than 80% (e.g., from about 80% to about 99%, from about 83% to about 99%, from about 85% to about 99%, from about 88% to about 99%, from about 90% to about 99%, from about 85% to about 96%, from about 88% to about 95%, etc.).

Further included herein are compositions and methods for culturing T cells under various conditions. In many instances such conditions will include the use of one or more of the following compositions: (1) OPTMIZER™ CTS™ SFM (Thermo Fisher Scientific, cat. no. A1048501), CTS™ Immune Cell Serum Replacement (ICSR) (Thermo Fisher Scientific, cat. no. A2596101), and/or a glutamine source may be one that will not form substantial amounts of ammonia (e.g., an L-alanyl-L-glutamine dipeptide). In many instances, such conditions will include the cultivation of cells under static conditions (e.g., in well of a stationary plate)), under conditions where the cell are kept in suspension (e.g., in a bag agitated on a rocker platform) and a combination of the two conditions (e.g., static for 1 day, followed by suspension for 9 days; static for 2 days, followed by suspension for 8 days; static for 4 days, followed by suspension for 6 days; suspension for 5 days, followed by static for 5 days, and suspension for 8 days, followed by static for 2 days.

In specific embodiments, provided herein are methods comprising the expansion of T cells in a culture vessels containing a gas permeable membrane (e.g., the G-REX® system), where the T cells are cultivated in a serum free medium (e.g., OPTMIZER™ CTS™ SFM or AIM-V® SFM, supplemented with CTS™ Immune Cell Serum Replacement (ICSR)) and, optionally, a glutamine source may be one that will not form substantial amounts of ammonia (e.g., an L-alanyl-L-glutamine dipeptide).

In some instances, the T cells will be polyclonal T cells. These T cells may be activated using CD3/CD28 beads, OKT3 mAb, virus-specific and tumor-specific T cells, etc.). Further such T cells may be gene-modified CAR T cells.

In some instances, culture methods are provided herein in which T cells expand at rate that is more rapid and/or to a higher cell density at a set time point than under another set of conditions. For example methods are provided herein where T cells expand more rapidly in suspension culture than in static culture. Specific methods include methods where T cells are expanded in a culture vessels containing a gas permeable membrane (e.g., the G-REX® system), where the T cells are cultivated in a OPTMIZER™ CTS™ SFM or AIM-V® SFM, supplemented with CTS™ Immune Cell Serum Replacement (ICSR). Further, such methods may be adjusted such the fold expansion after ten days is from about two fold to about ten fold (e.g., from about two fold to about eight fold, from about two fold to about six fold, from about two fold to about five fold, from about three fold to about eight fold, etc.) higher than under identical conditions except where the T cell are expanded in static culture (e.g., in a static bag or in the well of a microwell plate).

Culture Vessels with Gas Permeable Membranes

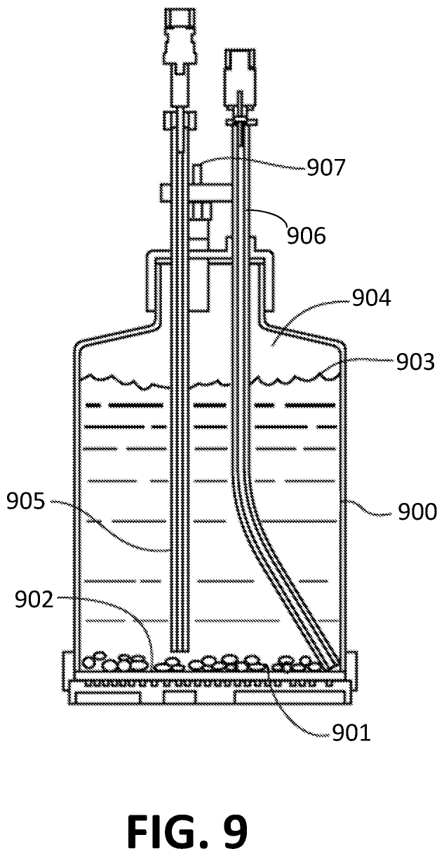

Exemplary culture vessels with gas permeable membranes are shown in FIG. 9. Such devices may be used to culture cells where the cells rest upon a gas permeable surface. Further, these cells may be maintained in a uniformly distributed state during expansion. In many instances, the gas permeable membrane is non-porous, liquid impermeable, and hydrophobic. Additional characteristics of gas permeable membranes that may be present in culture vessels used in methods set out herein are described elsewhere herein.

The gas permeable material preferably resides in a horizontal or substantially horizontal position during culture in order for cells to gravitate to the gas permeable material and distribute across the entire surface of the gas permeable material, and, when desired, in a uniform surface density (see FIG. 9). When the gas permeable membrane is located below the cells being cultured (see FIG. 9), it will be recognized that the weight of media the gas permeable material can move downward slightly in areas where it is not in direct contact with a support.

FIG. 9 shows an embodiment of a culture vessel with gas permeable membrane (900) that may be used in methods set out herein. Cells (901) rest upon a growth surface (902), which forms the bottom of the device and which is comprises a gas permeable membrane. The culture medium is located above the gas permeable membrane (903). Above the culture medium is located an air space (904). Further, a tube is located in the culture vessel for the removal of culture medium (905). An additional tube is located in the culture vessel for the addition of culture medium (906). The culture vessels also contain vent with a filter for maintaining sterility of the culture medium (907).

Specific culture vessels that may be used in methods set out herein include G-REX™ culture vessels marketed by Wilson Wolf Corporation, 33 5th Ave. NW, Suite 700, Saint Paul, Minn. 55112. Such vessels include those set out below in Table 2.

TABLE 2
G-REX ® Products

| Product Name | Part Number |
|---|---|
| G-REX ® 24 Well Plate | 80192M |
| G-REX6 ® Well Plate | 80240M |
| G-REX ® 6M Well Plate | 80660M |
| G-REX ® 10 | 80040S |
| G-REX100 ® | 80500 |
| G-REX ®100M | 81100 |
| G-REX ®100M-CS | 81100-CS |
| G-REX ®500M | 85500S |
| G-REX ®500M-CS | 85500S-CS |

Thus, methods set out herein include those in which immune cells are expanded in a cell culture vessels set out in Table 2. Further, immune cells may be expanded in well format culture vessels.

### T Cells

Any number of different types of T cells may be purified, isolated, activated and/or expanded by methods set out herein. Some of these T cells are as follows:

Naïve T cells are generally characterized by the surface expression of L-selectin (CD62L) and C—C Chemokine receptor type 7 (CCR7); the absence of the activation markers CD25, CD44 or CD69; and the absence of memory CD45RO isoform.

Th17 Cells: T helper 17 cells (or “Th17 cells” or “Th17 helper cells”) are an inflammatory subset of CD4+T helper cells that are believed to regulate host defense, and are involved in tissue inflammation and certain autoimmune diseases. It has been found that, when adoptively transferred into tumor-bearing mice, Th17 cells are more potent at eradicating melanoma than Th1 or non-polarized (ThO). The phenotype of Th17 cells is CD3+, CD4+, CD161+.

Memory T Cells: Memory T cells, also referred to as “antigen-experienced cells”, are experienced in a prior encounter with an antigen. These T cells are long-lived and can recognize antigens and quickly and strongly affect an immune response to an antigen to which they have been previously exposed. Memory T cells can include: stem memory cells (TSCM), central memory cells (TCM), effector memory cells (TEM). TSCM cells have the phenotype CD45RO−, CCR7+, CD45RA+, CD62L+(L-selectin), CD27+, CD28+ and IL-7Ra+, but they also express large amounts of IL-2R, CXCR3, and LFA-1. TCM cells express L-selectin and the CCR7, they secrete IL-2, but not IFNy or IL-4. TEM cells do not express L-selectin or CCR7 but produce effector cytokines like IFNy and IL-4.

Memory T cell subtypes: Central memory T cells (TCM cells) express CD45RO, C—C chemokine receptor type 7 (CCR7), and L-selectin (CD62L). Central memory T cells express intermediate to high levels of CD44. This memory subpopulation is commonly found in the lymph nodes, as well as in peripheral circulation.

Tissue resident memory T cells (TRM) occupy tissues (skin, lung, gastrointestinal tract, etc.) typically without recirculating. These cells are believed to play a role in protective immunity against pathogens. Dysfunctional TRM cells have been implicated in various autoimmune diseases.

Virtual memory T cells differ from the other memory subsets in that they do not appear to originate following a strong clonal expansion event. This population as a whole is typically abundant within the peripheral circulation.

### Treatment Methods

In an aspect is provided a method of treating a disease in a subject in need thereof, the method including administering to the subject T cells obtained by the method provided herein including embodiments thereof. Non-limiting examples of uses for CD8+ T cells (e.g., expanded populations of T cells comprising increased CD8+ T cell proportions, or CD8+ T cells isolated from such expanded populations) include: immunotherapies based on virus-specific T cells such as for cytomegalovirus (CMV) infection and for Epstein-Barr virus (EBV) infection for treatment of immunosuppressed transplant patients. See, e.g., Heslop et al. (2010) Blood 115(5):925-35. Additional non-limiting examples include the use of CAR-T and other modes of engineering virus-specific T cells for treatment of cancer and infectious disease. See, e.g., Pule et al. (2008) Nature Medicine 115(5):925-935 and Ghazi et al. (2013) J. Immunother. 35(2): 159-168. Non-limiting examples of uses for CD4+ T cells (e.g., expanded populations of T cells comprising increased CD4+ T cell proportions, or CD4+ T cells isolated from such expanded populations), include the treatment of HIV+ patients, and expanded CD4+T helper subsets (e.g., T_{H}1, T_{H}2, T_{H}3, T_{H}17, T_{H}9, or T_{FH}), and Regulatory T cells (Treg: CD4+CD25+FoxP3+) for treating autoimmunity. See, e.g., Tebas et al. (2014) N. Engl. J. Med. 370(10):901-10 and Riley et al. (2009) Immunity 30(5): 656-665.

In embodiments, the disease is a hyperproliferative disorder. In embodiments, the disease is an autoimmune disease. In embodiments, the disease is an inflammatory disease. In embodiments, the disease is an allergic disease. In embodiments, the disease is an infectious disease.

In embodiments, the infectious disease is a viral infection. In embodiments, the viral infection is a cytomegalovirus infection, an Epstein-Barr virus infection, or a human immunodeficiency virus infection.

In embodiments, the subject has a suppressed immune system. In embodiments, the subject has received a tissue or organ transplant. In embodiments, the subject has acquired immune deficiency syndrome.

In embodiments, the T cells are CD8+ T cells. In embodiments, the T cells are CD4+ T cells.

T cell subpopulations produced using the compositions and methods provided herein can be used in any number of physiological conditions, diseases and/or disease states for therapeutic purposes and/or research/discovery purposes. In embodiments, a condition or disease typified by an aberrant immune response is an autoimmune disease, for example diabetes, multiple sclerosis, myasthenia gravis, neuritis, lupus, rheumatoid arthritis, psoriasis, or inflammatory bowel disease. In embodiments, a condition in which immune suppression would be advantageous include conditions in which a normal or an activated immune response is disadvantageous to the mammal. In embodiments, the use of such cells before, during, or after transplantation avoids extensive chronic graft versus host disease which may occur in patients being treated (e.g., transplant patients). In embodiments, the cells may be expanded immediately after harvest or stored (e.g., by freezing) prior to expansion or after expansion and prior to their therapeutic use. In embodiments, such therapies may be conducted in conjunction with known immune suppressive therapies.

In embodiments, T cells are isolated based upon the stage of differentiation. T cell populations may be assessed for the stage of differentiation based upon the presence or absence of certain cellular markers or proteins. Markers used to assess the stage of T cell differentiation include: CD3, CD4, CD5, CD8, CD11c, CD14, CD19, CD20, CD25, CD27, CD33, CD34, CD45, CD45RA, CD45RB, CD56, CD62L, CD123, CD127, CD278, CD335, CD11a, CD45RO, CD57, CD58, CD69, CD95, CD103, CD161, CCR7, as well as the transcription factor FOXP3.

In embodiments, once an appropriate T cell population or sub population has been isolated from a patient or animal, genetic or any other appropriate modification or manipulation may optionally be carried out before the resulting T cell population is expanded using compositions and methods set out herein. The manipulation may, for example, take the form of stimulate/re-stimulation of the T cells with anti-CD3 and anti-CD28 antibodies to activate/re-activate them.

In embodiments, it may be desired to administer activated T cells to a subject and then subsequently redraw blood (or have an apheresis performed), activate and expand T cells therefrom according to a method provided herein, and reinfuse the patient with these activated and expanded T cells. This process can be carried out multiple times every few weeks. In embodiments, T cells can be expanded from blood draws of from 10 ml to 400 ml. In embodiments, T cells are expanded from blood draws of about 20 ml, about 30 ml, about 40 ml, about 50 ml, about 60 ml, about 70 ml, about 80 ml, about 90 ml, or about 100 ml. In embodiments, the administration of the subject compositions may be carried out in any convenient manner, including by aerosol inhalation, injection, ingestion, transfusion, implantation or transplantation.

In embodiments, a T cell subpopulation generated according to a method provided herein may have many potential uses, including experimental and therapeutic uses. In embodiments, a small number of T cells are removed from a patient and then manipulated and expanded ex vivo before reinfusing them into the patient. Non-limiting examples of diseases that may be treated in this way are autoimmune diseases and conditions in which suppressed immune activity is desirable (e.g., for allo-transplantation tolerance). In embodiments, a therapeutic method comprises providing a mammal, obtaining a biological sample from the mammal that contains T cells; expanding/activating the T cells ex vivo in accordance with the methods provided herein; and administering the expanded/activated T cells to the mammal to be treated. In embodiments, the first mammal and the mammal to be treated can be the same or different. In embodiments, the mammal can generally be any mammal, such as a cat, dog, rabbit, horse, pig, cow, goat, sheep, monkey, or human. In embodiments, the first mammal (“donor”) can be syngeneic, allogeneic, or xenogeneic.

In embodiments, T cell subpopulations produced using the compositions and methods provided herein can be used in a variety of applications and treatment modalities. In embodiments, T cell subpopulations can be used in the treatment of disease states including, but not limited to, cancer, autoimmune disease, allergic diseases, inflammatory diseases, infectious diseases, and graft versus host disease (GVHD). In embodiments, a T cell therapy includes infusion to a subject of T cell subpopulations externally expanded by methods provided herein following or not following immune depletion, or infusion to a subject of heterologous externally expanded T cells that have been isolated from a donor subject (e.g., adoptive cell transfer).

Autoimmune diseases or disorders are those diseases that result from an inappropriate and excessive response to a self-antigen. In embodiments, an autoimmune disorder comprises defective Treg cells. Non-limiting examples of autoimmune diseases include: diabetes mellitus, uveoretinitis and multiple sclerosis, Addison's disease, celiac disease, dermatomyositis, Grave's disease, Hashimoto's thyroiditis, alopecia areata, ankylosing spondylitis, autoimmune hepatitis, autoimmune parotitis, hemolytic anemia, pemphigus vulgaris, and psoriasis. In autoimmune disease states, the CD4⁺ CD25⁺ Tregs may be present in decreased number or be functionally deficient. Tregs from peripheral blood having reduced capacity to suppress T cell proliferation have been found in patients with multiple sclerosis (Viglietta et al., J. Exp. Med. 199:971-979 (2004).), autoimmune polyglandular syndrome type II (Kriegel et al., J. Exp. Med. 199:1285-1291 (2004).), type I diabetes (Lindley et al. Diabetes 54:92-929 (2005).), psoriasis (Sugiyama et al., J. Immunol. 174:164-173 (2005)), and myasthenia gravis (Balandina et al., Blood 105:735-741 (2005)).

In embodiments, treatment of autoimmune disorders with T cell therapy may involve differing mechanisms. In embodiments, blood or another source of immune cells can be removed from a subject inflicted with an autoimmune disorder. In embodiments, a method disclosed herein is used to expand T cell types other than memory T cells from the patient sample. In embodiments, following removal and expansion of autologous cells, inappropriate memory T cells can be depleted within a subject in need thereof by known methods, including low dose total body radiation, thymic irradiation, anti-thymocyte globulin, and administration of chemotherapy.

Alternatively, or in addition to the above described treatment modalities, Treg cells can be isolated from sources including peripheral blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. In embodiments, these T cells are expanded using methods provided herein. In embodiments, these expanded Treg cells can be re-administered to a patient to suppress inappropriate immune responses. In embodiments, this Treg therapy may be administered either to suppress the minimal remaining immune responses following immune depletion, or in subjects that have not undergone immune depletion.

In embodiments, a method of treating, reducing the risk of, or the severity of, an adverse GVHD event with T cell therapy is provided. In embodiments, a subject has GVHD. In embodiments, the GVHD follows hematopoietic stem cell transplantation. In embodiments, the GVHD is caused by alloreactive T cells present in the infused hematopoietic stem cell preparation. In embodiments, a subject has received organ transplantation and suffers or is at risk of suffering from graft rejection mediated by alloreactive host T cells. In embodiments, blood or another source of immune cells can be removed from a subject inflicted with GVHD. In embodiments, a method provided herein is used to selectively expand T cell types other than memory T cells, selectively expanding those cell types that do not comprise long-lasting recognition of antigens from the exogenous tissue. In embodiments, following removal and external expansion of autologous cells, inappropriate memory T cells can be depleted within a subject in need thereof by known methods, including low dose total body radiation, thymic irradiation, anti-thymocyte globulin, and administration of chemotherapy.

In embodiments, Treg cells removed from patient blood can be expanded. Further, these expanded Treg cells may readministered to a patient to suppress inappropriate immune responses, either to suppress the minimal remaining immune responses following immune depletion, or in subjects that have not undergone immune depletion.

Also provided herein are methods for treating inflammatory diseases and inflammation associated disorders. Many of these diseases can also be categorized as autoimmune disorders. Non-limiting examples of inflammatory diseases and inflammation associated disorders include: diabetes; rheumatoid arthritis; inflammatory bowel disease; familial Mediterranean fever; neonatal onset multisystem inflammatory disease; tumor necrosis factor (TNF) receptor-associated periodic syndrome (TRAPS); deficiency of interleukin-1 receptor antagonist (DIRA); and Bechet's disease.

Without being bound by any theory, because of the role of Treg cells in suppressing inappropriate immune responses to non-pathogenic antigens, decreased numbers or impaired functioning of these T cell subpopulations can contribute to inflammatory diseases. This is true of, for example, inflammatory bowel disease (Himmell et al., Immunology, 136:115-122 (2012)) and rheumatoid arthritis (Noack et al., Autoimmunity Reviews, 13:668-677 (2014)).

In embodiments, blood can be removed from a subject suffering from an inflammatory disorder. In embodiments, a method provided herein can be used to selectively expand non T memory cell T cell types, selectively expanding those cell types that do not comprise long-lasting recognition of inappropriate antigens (e.g., carbamylated proteins in anti-carbamylated protein (anti-CarP) antibody mediated rheumatoid arthritis). Following removal and expansion of autologous cells, inappropriate memory T cells can be depleted within a subject in need thereof by known methods, including low dose total body radiation, thymic irradiation, anti-thymocyte globulin, and administration of chemotherapy. Examples of chemotherapeutic agents include but are not limited to campath, anti-CD3 antibodies, cytoxin, fludarabine, cyclosporine, FK506, mycophenolic acid, steroids, FR901228, and irradiation.

Methods for treating hyperproliferative disorders (such as cancer) are also provided herein. In embodiments, increased Treg activity may result in poor immune response to tumor antigens and contribute to immune dysfunction. Elevated populations of CD4+CD25+ have been found in lung, pancreatic, breast, liver and skin cancer patients, in either the blood or tumor itself (Woo E Y, et al.; J Immunol 2002; 168:4272-6.; Wolf A M, et al. Clin Cancer Res 2003; 9:606-12.; Liyanage U K, et al. J Immunol 2002; 169:2756-61.; Viguier M, et al. J Immunol 2004; 173:1444-53.; Ormandy L A, et al. Cancer Res 2005; 65:2457-64.).

In embodiments, T cells specific for tumor antigens or hyperproliferative disorder antigens or antigens associate with a hyperproliferative disorder are expanded using a method or composition disclosed herein. Tumor antigens are proteins that are produced by tumor cells that elicit an immune response, particularly T cell mediate immune responses.

In embodiments, cancers that may be treated include tumors that are not vascularized, or not yet substantially vascularized, as well as vascularized tumors. In embodiments, the cancers may comprise non-solid tumors (such as hematological tumors, for example, leukemias and lymphomas) or may comprise solid tumors. Types of cancers to be treated include but are not limited to carcinoma, blastoma, and sarcoma, and certain leukemia or lymphoid malignancies, benign and malignant tumors, and malignancies, e.g., sarcomas, carcinomas, and melanomas.

Hematologic cancers are cancers of the blood or bone marrow. Non-limiting examples of hematological (or hematogenous) cancers include leukemias, including acute leukemias, chronic leukemias, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, and myelodysplasia.

Solid tumors are abnormal masses that usually do not contain cysts or liquid areas. Solid tumors can be benign or malignant. Different types of solid tumors are named for the types of cells that form them (such as sarcomas, carcinomas, and lymphomas). Non-limiting examples of solid tumors such as sarcomas and carcinoma, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, and sweat gland carcinoma.

In embodiments, expanded T cells are genetically modified the T cells to target antigens expressed on tumor cells through the expression of chimeric antigen receptors (CARs). In some embodiments, T cells that express CARs are expanded. CARs are antigen receptors that are designed to recognize cell surface antigens in a human leukocyte antigen independent manner. In some embodiments, immune cells may be collected from patient blood or other tissue. In embodiments, the T cells are engineered as described below to express CARs on their surface, allowing them to recognize specific antigens (e.g., tumor antigens). In embodiments, these CAR T cells can then be expanded by methods set out herein and infused into the patient. In embodiments, T cells are administered at 1×10⁵, 1×10⁶, 1×10⁷, 1×10⁸, 5×10⁸, 1×10⁹, 5×10⁹, 1×10¹⁰, 5×10¹⁰, 1×10¹¹, 5×10¹¹, or 1×10¹² cells to the subject. In embodiments, following patient infusion, the T cells will continue to expand and express the CAR, allowing for the mounting of an immune response against cells harboring the specific antigen the CAR is engineered to recognize.

In some embodiments, a cell (e.g., a T cell) engineered to express a CAR, wherein the CAR T cell exhibits an antitumor property, is provided. In embodiments, the CAR is be engineered to comprise an extracellular domain having an antigen binding domain fused to an intracellular signaling domain of the T cell antigen receptor complex zeta chain (e.g., CD3 zeta). In some embodiments, the CAR, when expressed in a T cell is able to redirect antigen recognition based on the antigen binding specificity.

In embodiments, the antigen binding moiety of the CAR comprises a target-specific binding element otherwise referred to as an antigen binding moiety. In embodiments, the choice of moiety depends on the type and number of ligands that define the surface of a target cell. For example, the antigen binding domain may be chosen to recognize a ligand that acts as a cell surface marker on target cells associated with a particular disease state. Thus the antigen moiety domain in the CAR may include, for example, those associated with viral, bacterial and parasitic infections, autoimmune disease and cancer cells.

In embodiments, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. In embodiments, cloning vectors contain transcription and translation terminators, initiation sequences, and promoters useful for regulation of the expression of the desired nucleic acid sequence.

In embodiments, the T cells may be used for nucleic acid immunization and gene therapy, using standard gene delivery protocols. Methods for gene delivery are known in the art. See, e.g., U.S. Pat. Nos. 5,399,346; 5,580,859; 5,589,466. In embodiment, a gene therapy vector is provided.

In embodiments, the nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

In embodiments, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

In embodiments, additional promoter elements (e.g., enhancers) regulate the frequency of transcriptional initiation. In embodiments, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Methods of making CAR T cells are known in the art (see, e.g., U.S. Pat. No. 8,906,682).

In embodiment, where a T cell is a CAR T cell, the selection of the antigen binding moiety may depend on the particular type of cancer to be treated. Tumor antigens are known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RUL RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, surviving and telomerase, prostate-carcinoma tumor antigen-1 (PCTA-1), MAGE, ELF2M, neutrophil elastase, ephrin B2, CD22, insulin growth factor (IGF-1), IGF-II, IGF-I receptor and mesothelin.

### Examples of Sources of Mixed Population of T Cells

In embodiments, the starting source for a mixed population of T cell is blood (e.g., circulating blood) which may be isolated from a subject. In embodiments, circulating blood can be obtained from one or more units of blood or from an apheresis or leukapheresis. In embodiments, the apheresis product typically contains lymphocytes, including T cells, monocytes, granulocytes, B cells, other nucleated white blood cells, red blood cells, and platelets. T cells can be obtained from a number of sources, including (but not limited to) blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. T cells can be obtained from T cell lines and from autologous or allogeneic sources. T cells may also be obtained from a xenogeneic source, for example, from mouse, rat, non-human primate, and pig.

In embodiments, T cells can be obtained from a unit of blood collected from a subject using any number of techniques known to the skilled artisan, such as Ficoll separation. T cells may be isolated from the circulating blood of a subject. In embodiments, blood may be obtained from the subject by apheresis or leukapheresis. In embodiments, the apheresis product typically contains lymphocytes, including T cells, monocytes, granulocytes, B cells, other nucleated white blood cells, red blood cells, and platelets. In embodiments, prior to exposure to a sensitizing composition and subsequent activation and/or stimulation, a source of T cells is obtained from a subject. In embodiments, the cells collected by apheresis may be washed to remove the plasma fraction and to place the cells in an appropriate buffer or media for subsequent processing steps. In embodiments set out herein, cells may be washed with phosphate buffered saline (PBS). In embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, Baxter) according to the manufacturer's instructions. In embodiments, after washing, the cells may be resuspended in a variety of biocompatible buffers, such as, for example, calcium (Ca)-free, magnesium (Mg)-free PBS. In embodiments, the undesirable components of the apheresis sample may be removed and the cells directly resuspended in culture media.

In embodiments, T cells are isolated from peripheral blood lymphocytes by lysing or removing the red blood cells and depleting the monocytes, for example, by centrifugation through a PERCOLL™ gradient. In embodiments, a specific subpopulation of T cells can be further isolated by positive or negative selection techniques.

In embodiments, T cells can be positively selected for CD3+ cells. Any selection technique known to one of skill in the art may be used. One non-limiting example is flow cytometric sorting. In another embodiment, T cells can be isolated by incubation with anti-CD3 beads. One non-limiting example is anti-CD3/anti-CD28-conjugated beads, such as DYNABEADS® Human T-Expander CD3/CD28 (Life Technologies Corp., Cat. No. 11141D), for a time period sufficient for positive selection of the desired T cells. In embodiments, the time periods ranges from 30 minutes to 36 hours or longer and all integer values there between. In embodiments, the time period is at least 1, 2, 3, 4, 5, or 6 hours. In another embodiment the time period is 10 to 24 hours. In embodiments, the incubation time period is 24 hours. Longer incubation times, such as 24 hours, can increase cell yield. In embodiments, longer incubation times may be used to isolate T cells in any situation where there are few T cells as compared to other cell types. In embodiments, enrichment of a T cell population by negative selection can be accomplished with a combination of antibodies directed to surface markers unique to the negatively selected cells. One possible method is cell sorting and/or selection via magnetic immunoadherence or flow cytometry that uses a cocktail of monoclonal antibodies direct to cell surface markers present on the cells negatively selected. In embodiments, the fold expansion may differ based on the starting materials due to the variability of donor cells. In embodiments, the normal starting density can be between about 0.5×10⁶ to about 1.5×10⁶.

In embodiments, T cell subpopulations may be generated by selection on the basis of whether one or more marker(s) is/are present or absent. For example, Treg cells may be obtained from a mixed population based upon the selection of cells that are CD4+, CD25+, CD127neg/low and, optionally, FOXP3+. In embodiments, Treg cells may be FOXP3-. Selection, in this instance, effectively refers to “choosing” of the cells based upon one or more definable characteristic. Further, selection can be positive or negative in that it can be for cells have one or more characteristic (positive) or for cells that do not have one or more characteristic (negative).

With respect to Treg cells, for purposes of illustration, these cells may be obtained from a mixed population through the binding of these cells to a surface (e.g., magnetic beads) having attached thereto antibodies that bind to CD4 and/or CD25 and the binding of non-Treg cells to a surface (e.g., magnetic beads) having attached thereto antibodies that binding CD127. As a specific example, magnetic beads having bound thereto an antibody that binds to CD3 may be used to isolate CD3+ cells. Once released, CD3+ cells obtained may then be contacted with magnetic beads having bound thereto an antibody that binds to CD4. The resulting CD3+, CD4+ cells may then be contacted with magnetic beads having bound thereto an antibody that binds to CD25. The resulting CD3+, CD4+, CD25+ cells may then be contacted with magnetic beads having bound thereto an antibody that binds to CD127, where the cells that are collected are those that do not bind to the beads.

In embodiments, multiple characteristics may be used simultaneously to obtain a T cells subpopulation (e.g., Treg cells). For example, a surface containing bound thereto antibodies that bind to two or more cell surface marker may also be used. As a specific example, CD4+, CD25+ cells may be obtained from a mixed population through the binding of these cells to a surface having attached thereto antibodies that bind to CD4 and CD25. The selection for multiple characteristics simultaneously may result in number of undesired cells types “co-purifying” with the desired cell type(s). This is so because, using the specific example above, cells that are CD4+, CD25- and CD4-, CD25+ may be obtained in addition to CD4+, CD25+ cells.

Flow cytometry is particularly useful for the separation of cells based upon desired characteristics. Cells may be separated based upon detectable labels associated with molecules that bind to cells of interested (e.g., a natural ligand such as IL-7 binding to CD127, an antibody specific for CD25, etc.). Thus, ligands that bind to cellular components that may be detected and/or differentiated by flow cytometry systems may be used to purify/isolate T cells that have specific characteristics. Further, the presence or absence of multiple characteristics may be simultaneously determined by flow cytometry.

Included herein are methods for obtaining members of one or more T cell subpopulations, where members of the T cell subpopulations are identified by specific characteristics and separated from cells with differ with respect to these characteristics. Examples of characteristics that may be used in methods set out herein include the presence or absence of the following proteins CD3, CD4, CD5, CD8, CD11c, CD14, CD19, CD20, CD25, CD27, CD33, CD34, CD45, CD45RA, CD56, CD62L, CD123, CD127, CD278, CD335, CCR7, K562P, K562CD19, and FOXP3.

### CAR-T Cells

Also provides are compositions and methods for making an comprising Chimeric antigen receptor T cells (CAR T cells). Chimeric antigen receptors (CARs) are engineered receptors designed to provide a designated an immune effector cell. The receptors are called chimeric because they are composed of parts from different sources.

In many instances, CAR T cells express recombinant receptors that combine antigen-binding and T-Cell activating functions. Typically CARs contain three regions: An extracellular domain, a transmembrane domain, and an intracellular domain.

The extracellular domain is the region of the receptor that is exposed to the exterior of the cell and if typically contains three regions: a signal peptide, an antigen recognition region, and a spacer. The signal peptide facilitates integration of the CAR into the cell membrane. The antigen recognition region of CARs are typically single-chain variable antibody fragment (e.g., an antibody fragment with binding activity for the CD19 receptor). The transmembrane domain (e.g., CD28 transmembrane domain) is typically a hydrophobic region that spans the T cell's cell membrane and allows for passage of signals received by the extracellular domain to be transmitted into the interior of the T cell. After antigen recognition, receptors cluster and a signal is transmitted to intracellular domain.

Nucleic acid molecules encoding CARs may be structured in any number of formats and may be introduced into T cells by any number of methods. CAR coding regions will normally be operably linked to expressions control sequences, such as a promoter (e.g., a CMV promoter). Further, these nucleic acid molecules will typically be present in a nucleic acid vector (e.g., a cloning vector) containing components such as elements for regulated, translation terminator, and one or more selectable markers.

One approach to treating subjects in need thereof or patients is to use the expanded T cells and genetically modify the T cells to target antigens expressed on tumor cells through the expression of CARs. In many instances, nucleic acid molecules encoding proteins, such as a CAR, will be introduced into T cells, followed by expansion of the engineered T cells.

In treatment utilizing CARs immune cells may be collected from patient blood or other tissue. The T cells are engineered as described below to express CARs on their surface, allowing them to recognize specific antigens (e.g., tumor antigens). These CAR T cells can then be expanded by methods set out herein and infused into the patient. Following patient infusion, the T cells will continue to expand and express the CAR, allowing for the mounting of an immune response against cells harboring the specific antigen the CAR is engineered to recognize.

Also provided herein are cells (e.g., T cells) engineered to express a CAR wherein the CAR T cell exhibits an antitumor property. The CAR may be designed to comprise an extracellular domain having an antigen binding domain fused to an intracellular signaling domain of the T cell antigen receptor complex zeta chain (e.g., CD3 zeta). The CAR, when expressed in a T cell is able to redirect antigen recognition based on the antigen binding specificity.

The antigen binding moiety of the CAR comprises a target-specific binding element otherwise referred to as an antigen binding moiety. The choice of moiety depends on the type and number of ligands that define the surface of a target cell. For example, the antigen binding domain may be chosen to recognize a ligand that acts as a cell surface marker on target cells associated with a particular disease state. Thus, the antigen moiety domain of CARs includes those associated with viral, bacterial and parasitic infections, autoimmune disease and cancer cells.

The expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements (e.g., enhancers) regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Methods of making CAR T cells are known in the art (see, e.g., U.S. Pat. No. 8,906,682).

### Kits of the Invention

Also provided herein are kits comprising (i) compositions for the isolation of T cells from a subject; (ii) compositions for the ex vivo culture of T cells and (iii) compositions for the selective expansion of one or more T cell subpopulation (e.g., Th17, regulatory T cells (Treg cells), memory T cells, etc.).

Kits may include one or more component used in methods setout herein. Such components include (1) one or more culture more, (2) one or more culture medium supplement, (3) one or more protein (e.g., one or more cytokine, one or more chemokine, one or more serum albumin), and/or (4) one or more culture vessel (e.g., one or more culture vessel with a gas permeable membrane).

Kits can also include written instructions for use of the kit, such as instructions for wash steps, culturing conditions and duration of incubation of isolated T cells with compositions set out herein for the selective expansion of specific T cell subpopulations.

### EXAMPLES

The following examples illustrate certain specific embodiments and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Human T Cell Expansion Ex Vivo Using Serum-Free Media and Gas-Permeable Rapid Expansion (G-REX®) Cell Culture Devices

### Introduction

Adoptive immunotherapy with ex vivo-modified T cells shows immense promise as an emerging strategy for patients with advanced malignancies. Although promising, most current methods for expansion of gene-modified T cells ex vivo are complicated and labor intensive, limiting the broad application of adoptive immunotherapy in the future.

Traditional T cell expansion protocols are marked by flaws in consistency, safety, the frequency of human intervention required, and the length of the manufacturing process. These expansion protocols have traditionally involved the use human serum, which is characterized by batch inconsistency, and can potentially expose the patient to adverse viral contamination. Moreover, the duration of the expansion phase has typically been 10-12 days and involves extensive hands-on operation, while an ideal process would be significantly shorter and require minimal cellular disturbance.

For the purpose of safety and consistency, serum-free T cell expansion media have been developed, and to minimize complexity of the manufacturing process, many researchers in the field are turning to the Gas Permeable Rapid Expansion (G-REX®) culture platform, which has shown superior cell output and a reduction in the number of required technician manipulations compared to conventional approaches. However, to date, little work has been done regarding the use of serum-free media in G-REX® systems. To this end, several commercially available serum-free media we tested to see how effectively they could expand human T cells in the G-REX® culture system. The results demonstrated that none of the serum-free media consistently performed as well as conventional methods that employ serum-containing culture media. However, when the best performer from this cohort of serum-free media was supplemented with 4 mM GLUTAMAX™ (Thermo Fisher Scientific, cat. no. 35050061) and 2% of a chemically defined serum replacement, this medium supported ample T cell expansion, with yields similar to or better than media containing human serum.

Furthermore, the resultant cell population displayed a higher frequency of the desirable central memory phenotype than the cells grown in serum-containing media and was indistinguishable from the serum-grown population with regards to both CD8/CD4 ratio and functionality. The combination of serum-free media with the G-REX® culture platform can be effective for human T cell expansion and that applying this cell culture strategy to the production of T cell therapies could potentially address some of the concerns associated with traditional protocols by ensuring safety and consistency, shortening the expansion phase, and reducing the excessive amount of technical intervention required.

The G-REX® system has been shown to support higher cell density per surface area than standard plate based culture systems. (Vera et al., J. Immunother. 33:305-315 (2010).) The G-Rex system has also been shown to be able to support and increased the rate of cell expansion and higher cell densities as compared to plate based culture.

### Materials and Methods

T Cell Isolation: Primary human T cells from normal donors were negatively isolated from PBMCs with DYNABEADS™ UNTOUCHED™ Human T Cells kits (Thermo Fisher Scientific, cat. no. 11344D), which can be used to remove cells having the following markers: CD14, CD16 (a and b), CD19, CD36, CD56, CD123 and CD235A (e.g., B cells, NK cells, monocytes, platelets, dendritic cells, granulocytes and erythrocytes).

Media: Basal growth media included X-VIVO 15™ (Lonza, cat. nos. BE02-060Q), OPTMIZER™ CTS™ SFM, AIM-V SFM, and RPMI 1640 (Thermo Fisher Scientific, cat. nos. A1048501, 0870112DK, 11875119). Media were supplemented with 5% human AB serum (hABs) (Gemini Bio-Products) or 2.5% CTS Immune Cell Serum Replacement (Thermo Fisher Scientific) where indicated.

Seeding: 1). For all 6-well G-REX® (Wilson-Wolf, cat #80240M) experiments, 5×10⁶ T cells were seeded in 100 ml of the indicated medium. 2). For static plate (12- and 24-well, Corning® Costar®, cat #CLS3513 and CLS3527, respectively) and static PL30 bag (Origen Biomedical, cat #PL30-2G) experiments, T cells were seeded at 1×10⁶ cells/ml of the indicated medium.

Activation: 1). For all G-REX® and plate/flask experiments, T cells were activated with DYNABEADS™ Human T-Expander CD3/CD28 (Thermo Fisher Scientific, cat. no. 11141D) at a. ratio of 3 beads per T cell in the presence of 100 IU/ml of rIL-2 (Thermo Fisher Scientific, cat. no. PHC0021). 2). For bag experiments, T cells were activated using 50 ng/ml of soluble anti-CD3 (eBioscience, cat. no. 16-0037-85) in the presence of 300 IU/ml of rIL-2.

Expansion: T cells were maintained at 5×10⁶ cells/ml and counted on days 3, 5, 7, and 10 using a Beckman-Coulter Vi-Cell analyzer. In addition, rIL-2 was replenished on these same days. Cell growth is expressed as fold expansion over time. For cells grown in G-REX® culture devices, media was exchanged on days 5 and 7 and 100 IU/ml of rIL-2 was replenished on days 3, 5, and 7. The vessels were incubated at 37° C., and a relative humidity of about 95 percent. The CO₂ concentration was about 5 percent. The O₂ concentration was about 17-21%.

Endpoints: Cellular phenotype was assessed on day 10 by staining T cells with anti-CD3-Pacific Orange, anti-CD4-FITC, anti-CD8-Pacific Blue, anti-CD62L-APC, and anti-CCR7-PE (Thermo Fisher Scientific, cat. nos. CD0330, 11-0041-82, MHCD0828, 17-0621-82, 12-1971-82). To assess cytokine production (data not shown), DYNABEADS™ Human T-Expander CD3/CD28 we removed from the cultures on day 10, washed the T cells, and rested them overnight in fresh medium. 2.5 million T cells were seeded at 1×10⁶ T cell/mL and re-stimulated with Human T-Expander CD3/CD28 at a 1:1 bead to cell ratio and incubated for 24 hours. Supernatants were collected and processed for analysis with INVITROGEN™ Cytokine Human Magnetic 35-Plex Panel for LUMINEX™ (Thermo Fisher Scientific, cat. no. LHC6005M).

### Results

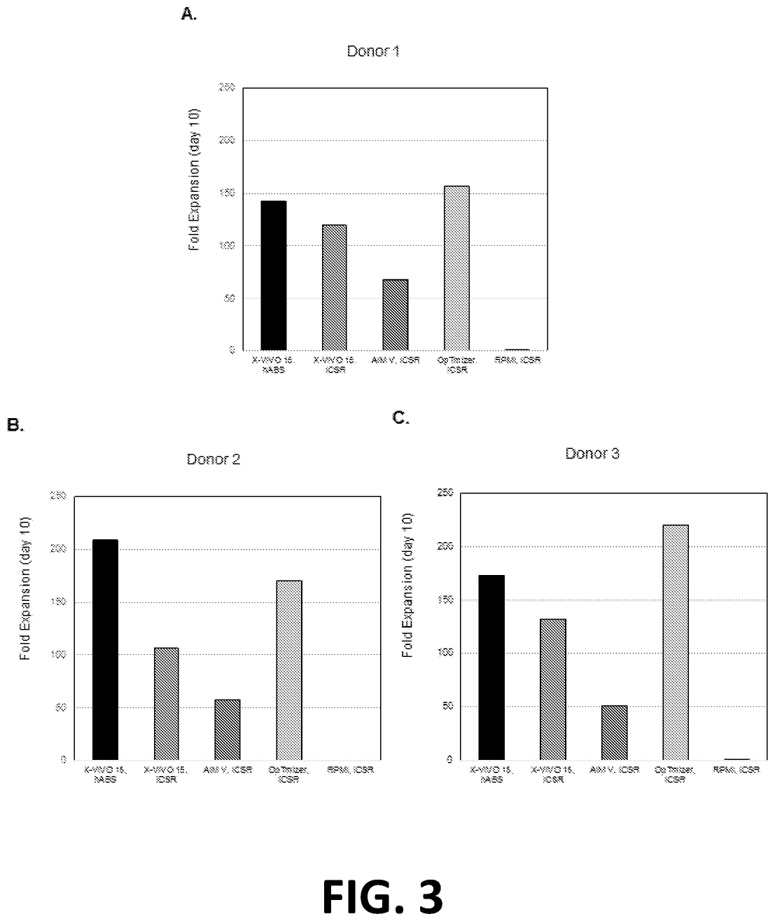

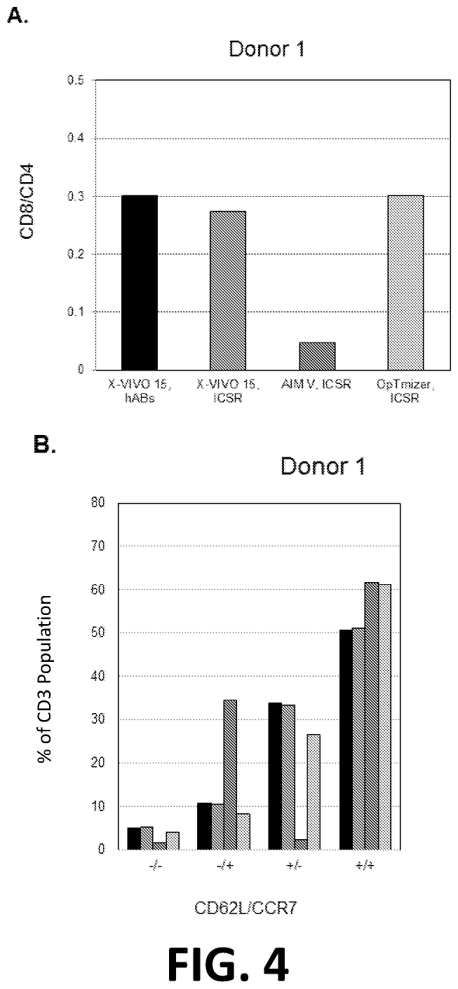

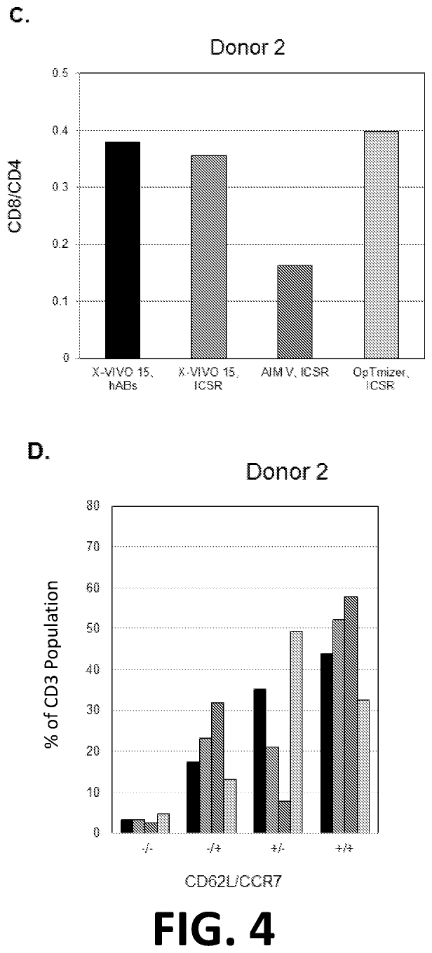

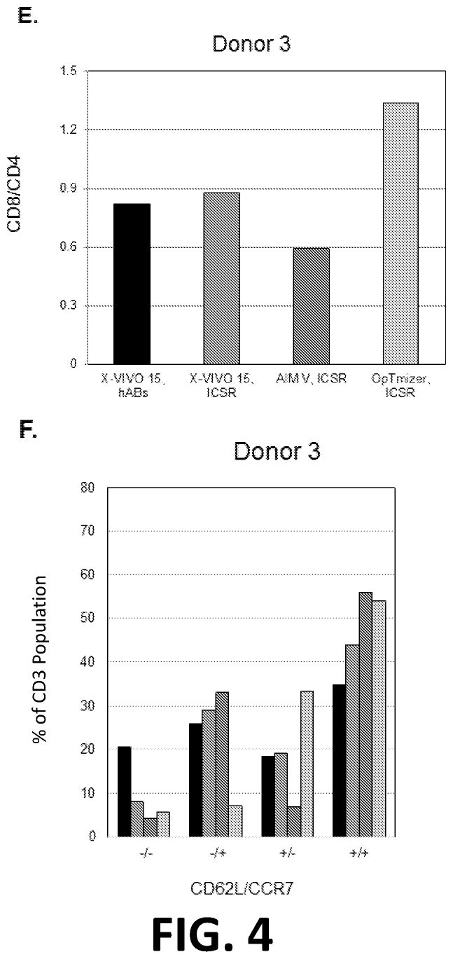

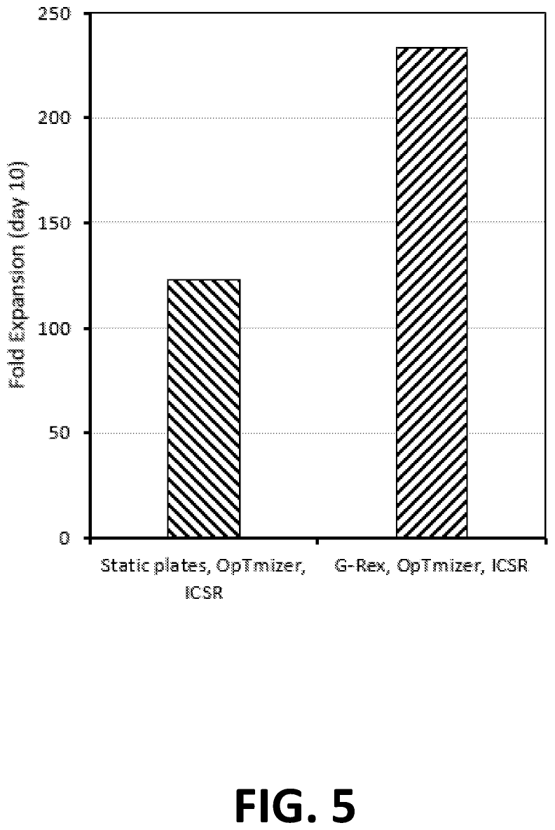

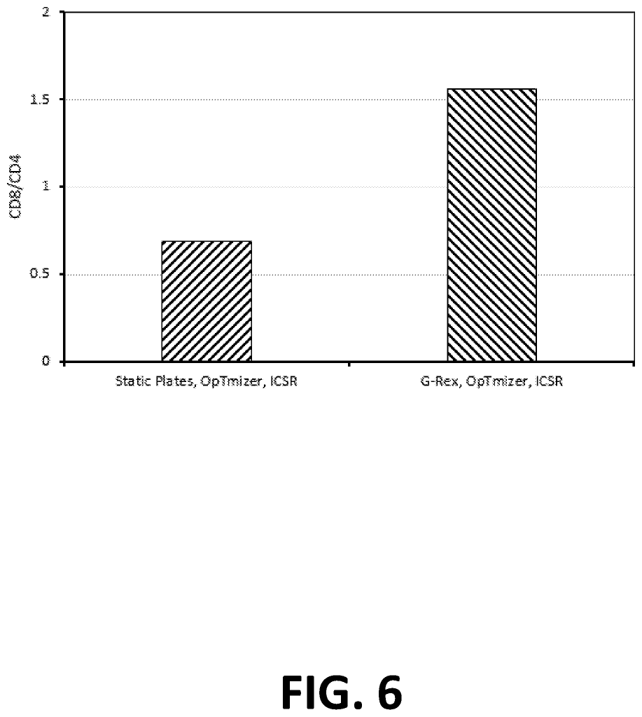

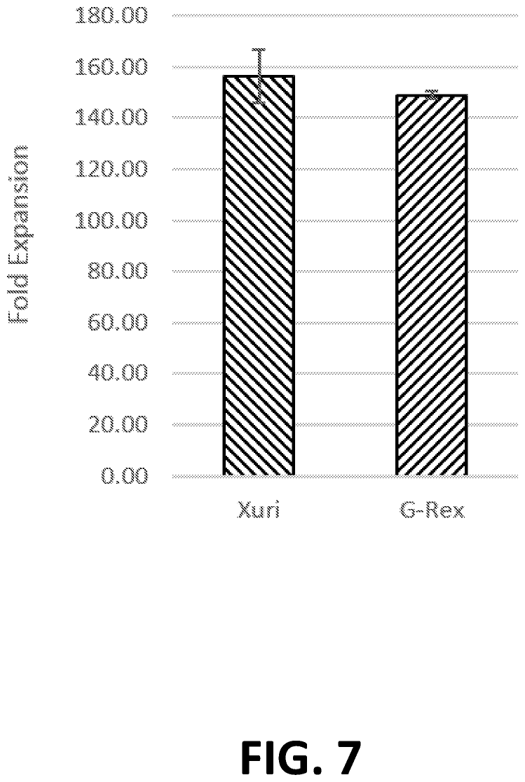

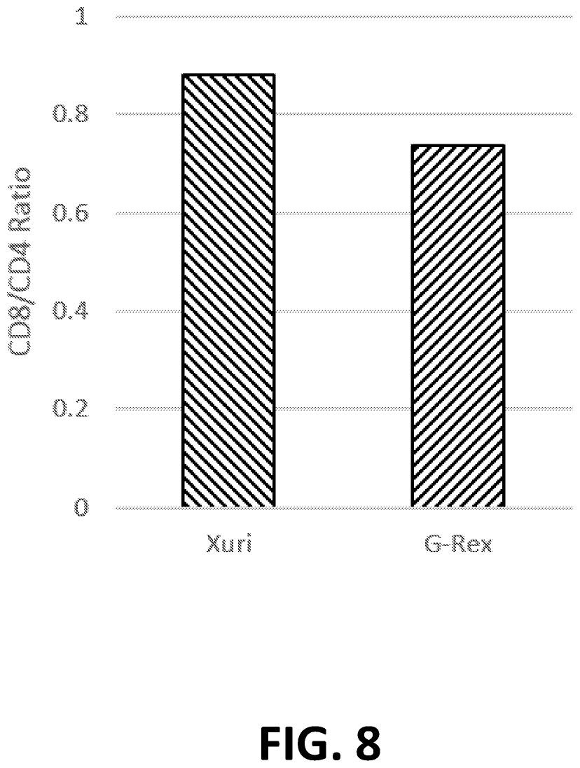

TABLE 3
Cell culture growth characteristics of T cells expanded in the G-REX ® system
in OPTMIZER ™ and ICSR (see FIG. 3).

| | | Vol. | | Increase | Fold | Population | Viability |
|---|---|---|---|---|---|---|---|
| Days | Cells/ml | (ml) | Total Cells | Factor | Expansion | Doublings | (%) |
| 0 | 4.55 × 10⁴ | 110 | 5.00 × 10⁶ | N/A | 0.00 | 0 | 88.5 |
| 3 | 1.20 × 10⁵ | 25 | 3.00 × 10⁶ | 0.6 | 0.60 | −0.740 | 100 |
| 4 | 7.50 × 10⁵ | 41 | 3.08 × 10⁷ | 10.25 | 6.15 | 2.621 | 95.00 |
| 5 | 1.81 × 10⁶ | 38 | 6.88 × 10⁷ | 2.237 | 13.76 | 3.782 | 82.1 |
| 6 | 3.94 × 10⁶ | 38 | 1.50 × 10⁸ | 2.177 | 29.94 | 4.904 | 84.1 |
| 7 | 4.92 × 10⁶ | 41 | 2.02 × 10⁸ | 1.347 | 40.34 | 5.334 | 75.70 |
| 8 | 4.42 × 10⁶ | 44.5 | 1.97 × 10⁸ | 0.975 | 39.34 | 5.298 | 66.60 |
| 9 | 4.15 × 10⁶ | 43 | 1.78 × 10⁸ | 0.907 | 35.69 | 5.158 | 59.70 |
| 10 | 4.50 × 10⁶ | 44 | 1.98 × 10⁸ | 1.110 | 39.60 | 5.307 | 54.1 |

### Conclusions

The addition of CTS™ Immune Cell Serum Replacement to serum-free media results in a more robust T cell expansion in the G-REX® platform. Supplementing serum-free media with CTS™ Immune Cell Serum Replacement for T cell expansion in the G-REX® culture vessel can lead to growth that is comparable to what is observed using serum-containing media. Cells grown in the G-REX® platform using serum-free media containing CTS™ Immune Cell Serum Replacement exhibit a desirable central memory phenotype. Serum-free media supplemented with CTS™ Immune Cell Serum Replacement can support T cell expansion in static culture bags and plates. Serum-free media with CTS™ Immune Cell Serum Replacement can support T cell expansion in rocking bioreactors (data not shown). T cells grown in serum-free media containing CTS™ Immune Cell Serum Replacement produce a cytokine profile that is similar to that of cells grown in serum-containing media (data not shown).

### Example 2: Human T Cell Expansion Ex Vivo Under Varying Conditions in OPTIMZER™ CTS™ SFM, Supplemented with 2.5% CTS™ Immune Cell Serum Replacement

Data set out in Tables 4A through 5C were generated using T cell cultures in OPTMIZER™ CTS™ SFM, supplemented with 2.5% CTS™ Immune Cell Serum Replacement. Methods used to generate the data set out in Tables 4A through 5C were generally as set out above in Example 1 isolation, media, seeding, G-REX®/plate activation, and the endpoints tested, with the following exceptions in bag activation and expansion:

Activation and Stimulation in all static vessels for Example 2: T cells were activated with DYNABEADS™ Human T-Expander CD3/CD28 (ThermoFisher Scientific, cat. no. 11141D) at a ratio of 3 beads per T cell in the presence of 100 IU/ml of rIL-2 for plate and G-REX® experiments and 300 IU/ml of rIL-2 for bag experiments.

Static Expansion: For static plates and bags, T cells were maintained at 5×10⁵ cells/ml and counted on days 3, 5, 7, and 10 using a Beckman-Coulter Vi-Cell analyzer. For G-REX® vessels, 20 ml of medium was swapped on days 5 and 7. For all conditions, 100 IU/ml of rIL-2 was replenished on days 3, 5, and 7.

GE XURI™ W25 Work Flow: Cells were activated at 1×10⁶ cells/ml with DYNABEADS™ Human T-Expander CD3/CD28 (Thermo Fisher Scientific, cat. no. 11141D) at a ratio of 3 beads per T cell in the presence of 300 IU/ml of rIL-2 in static PL240 bags (Origen Biomedical, cat. no. PL240-2G) for days 0-3. On day 3, the cells were inoculated into 1 L XURI™ Cellbag (GE, 2 L perf/DO/pH, cat. no. 29279164) at a density of 0.25×10⁶ cells/ml in 1 L of the indicated expansion medium containing 100 IU/ml of rIL-2. Dissolved oxygen was maintained at 30% using automated gas control and the pH was maintained ˜7 using perfusion. Perfusion of 500 ml/day was started on day 5 and the flow rate was increased to 1 L/day on day 7. Agitation speed was 12 rpms and rocker angle was 6°. Cell growth and viability were monitored daily and phenotype was assessed at day 10 using flow cytometry.

TABLE 4A
Cultured T Cells in G-REX ® and Static Bags at Day 10 (n = 2)
Fold Expansion

| | Bags | G-REX ® | | Bags | G-REX ® |
|---|---|---|---|---|---|
| Donor1 | 24.8 | 149.45 | Avg./ | 52.37/ | 151.03/ |
| Donor2 | 79.94 | 152.6 | SD | 38.99 | 2.23 |

TABLE 4B
CD4/CB8 Phenotype Day 10 (n = 2)

| | | | | | % CD4 | % CD8 | CD8/CD4 |
|---|---|---|---|---|---|---|---|
| | | % CD4 | % CD8 | CD8/CD4 | Avg | Avg | Avg |
| Donor1 | Bags | 77.34 | 11.36 | 0.147 | 53.385 | 33.32 | 1.01 |
| | G-REX ® | 74.7 | 18.38 | 0.246 | 55.38 | 36.885 | 0.89 |
| Donor2 | Bags | 29.43 | 55.28 | 1.878 | | | |
| | G-REX ® | 36.06 | 55.39 | 1.536 | | | |

TABLE 4C
CCR7/CD62L Phenotype at Day 10 (n = 2)

| | | % of CD3+: CCR7/CD62L | | | | | % of CD3+: CCR7/CD62L | | | |
|---|---|---|---|---|---|---|---|---|---|---|
| | | −/− | −/+ | +/− | +/+ | Avg | −/− | −/+ | +/− | +/+ |
| Donor1 | Bags | 31.13 | 5.15 | 26.33 | 37.39 | Bags | 20.75 | 3.32 | 23.11 | 52.83 |
| | G-REX ® | 33.14 | 2.33 | 26.77 | 37.76 | G-REX ® | 28.45 | 1.97 | 25.64 | 43.95 |
| Donor2 | Bags | 10.37 | 1.48 | 19.89 | 68.26 | | | | | |
| | G-REX ® | 23.75 | 1.6 | 24.5 | 50.14 | | | | | |

TABLE 5A
Cultured T Cells in G-REX ® and XURI ™ at Days 7 and 10 (n = 2)
Fold Expansion

| | Days | XURI ™ | G-REX ® | | Bags | G-REX ® |
|---|---|---|---|---|---|---|
| Donor1 | 0 | 0 | 0 | Avg | 0.00 | 0.00 |
| | 7 | 62.92 | 51.72 | | 55.96 | 50.60 |
| | 10 | 148.85 | 150.25 | | 156.38 | 149.12 |
| Donor2 | 0 | 0 | 0 | SD | 0.00 | 0.00 |
| | 7 | 49.01 | 49.47 | | 9.84 | 1.59 |
| | 10 | 163.90 | 147.99 | | 10.64 | 1.59 |

TABLE 5B
CD4/CB8 Phenotype Day 10 (n = 2)

| | | | | | % CD4 | % CD8 | CD8/CD4 |
|---|---|---|---|---|---|---|---|
| | | % CD4 | % CD8 | CD8/CD4 | Avg/SD | Avg/SD | Avg |
| Donor1 | XURI ™ | 61.9 | 36.52 | 0.59 | 50.52/16.09 | 44.62/11.46 | 0.88 |
| | G-REX ® | 57.83 | 40.16 | 0.69 | 52.85/7.04 | 38.97/1.68 | 0.74 |
| Donor2 | XURI ™ | 39.14 | 52.73 | 1.35 | | | |
| | G-Rex | 47.87 | 37.78 | 0.79 | | | |

TABLE 5C
CCR7/CD62L Phenotype at Day 10 (n = 2)

% of CD3+: CCR7/CD62L

| | −/− | −/+ | +/− | +/+ |
|---|---|---|---|---|
| XURI ™ | 11.63 | 6.12 | 49.88 | 32.37 |
| G-REX ® | 4.78 | 1.17 | 69.34 | 24.71 |

TABLE 6A
Cultured T Cells G-REX ® and Plates at Day 10 (n = 3)
Fold Expansion

| | Plates | G-REX ® | | Plates | G-REX ® |
|---|---|---|---|---|---|
| Donor1 | 160.71 | 218.95 | Avg. | 122.72 | 233.38 |
| Donor2 | 94.15 | 124.8 | | | |
| Donor3 | 113.31 | 356.38 | | | |

TABLE 6B
CD4/CB8 Phenotype Day 10 (n = 3)

| | | | | % CD4 | % CD8 | CD8/CD4 |
|---|---|---|---|---|---|---|
| | | % CD4 | % CD8 | Avg | Avg | Avg |
| Donor1 | Plate | 44.76 | 37.03 | 56.99% | 39.25% | 0.69 |
| | G-REX ® | 43.11 | 44.72 | 32.70% | 50.89% | 1.56 |
| Donor2 | Plate | 67.94 | 24.95 | | | |
| | G-REX ® | 44.13 | 44.31 | | | |
| Donor3 | Plate | 58.28 | 36.11 | | | |
| | G-REX ® | 30.52 | 63.65 | | | |

TABLE 6C
CCR7/CD62L Phenotype at Day 10 (n = 3)

| | | % of CD3+: CCR7/CD62L | | | | | % of CD3+: CCR7/CD62L | | | |
|---|---|---|---|---|---|---|---|---|---|---|
| | | −/− | −/+ | +/− | +/+ | Avg | −/− | −/+ | +/− | +/+ |
| Donor1 | Plate | 5.02 | 0.1 | 93.57 | 1.32 | Plate | 4.8 | 0.1 | 94.18 | 0.92 |
| | G-REX ® | 4.61 | 0.1 | 91.47 | 3.82 | G-REX ® | 3.75 | 0.1 | 92.72 | 3.44 |
| Donor2 | Plate | 5.37 | 0.19 | 93.43 | 1.02 | | | | | |
| | G-REX ® | 3.91 | 0.12 | 91.32 | 4.66 | | | | | |
| Donor3 | Plate | 4.02 | 0.02 | 95.55 | 0.42 | | | | | |
| | G-REX ® | 2.73 | 0.08 | 95.36 | 1.83 | | | | | |

### Other Embodiments

While the invention has been described in conjunction with the detailed description thereof, the foregoing description is intended to illustrate and not limit the scope of the subject matter described herein, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. GenBank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

Exemplary Subject Matter of the Invention is Represented by the Following Clauses:

Clause 1. A method for culturing T cells, the method comprising culturing the T cells under conditions where the T cells have a peak population maximum doubling time of from about 25 hours to about 40 hours, and wherein the T cells are cultured without serum.

Clause 2. The method of clause 1, wherein the T cells are cultured in the presence of serum albumin

Clause 3. The methods of clause 2, wherein the serum albumin is human serum albumin.

Clause 4. The methods of clauses 2 or 3, wherein the serum albumin is recombinant serum albumin

Clause 5. The methods of clauses 2 to 4, wherein the recombinant serum albumin is produced in a cell which is not a mammalian cell.

Clause 6. The methods of clauses 1 to 5, wherein T cells are cultured in a culture medium comprising OPTMIZER™ CTS™ SFM (Thermo Fisher Scientific, cat. no. A1048501).

Clause 7. The methods of clause 1, wherein T cells are cultured in a culture medium comprising CTS™ Immune Cell Serum Replacement (ICSR) (Thermo Fisher Scientific, catalog number A2596101).

Clause 8. The methods of clause 1, wherein T cells are cultured in a culture medium comprising OPTMIZER™ CTS™ SFM (Thermo Fisher Scientific, cat. no. A1048501) and CTS™ Immune Cell Serum Replacement (ICSR) (Thermo Fisher Scientific, catalog number A2596101).

Clause 9. The method of clauses 1 to 8, wherein T cells are cultured in an incubator where the O₂ concentration is between 15% and 25%.

Clause 10. The method of clauses 1 to 9, wherein T cells are cultured in an incubator where the CO₂ concentration is between 3% and 7%.

Clause 11. The method of clauses 1 to 5, wherein T cells are cultured at a temperature between 34° C. and 40° C.

Clause 12. The method of clauses 1 to 11, wherein T cells are cultured in the presence of a gas permeable membrane.

Clause 13. The method of clauses 1 to 11, wherein the T cells are cultured in a G-REX® culture vessel.

Clause 14. The method of clause 13, wherein the G-REX® culture vessel is G-REX® 6M Well Plate (Wolf Wilson Corporation, part number 80660M).

Clause 15. The method of clause 12, wherein the gas permeable membrane comprises silicone and is 0.005 to 0.007 inches thick.

Clause 16. The method of clauses 1 to 15, wherein the T cells are cultured in the presence of a glutamine source that will not form substantial amounts of ammonia.

Clause 17. The method of clause 16, wherein the glutamine source is an L-alanyl-L-glutamine dipeptide.

Clause 18. The method of clauses 1 to 17, wherein the T cells are present in a mixed population of different T cell subtypes.

Clause 19. The method of clauses 1 to 18, wherein the T cells reach a population density of between 1.0×10⁷ and 8.0×10⁷ cells per cm².

Clause 20. The method of clauses 1 to 19, wherein the T cells are obtained from a sample provided by a donor.

Clause 21. The method of clause 20, wherein the donor is a human donor.

Clause 22. The method of clauses 1 to 21, wherein the T cells are contacted with one or more agents that bind to one or more cell receptors present on the T cells.

Clause 23. The method of clause 22, wherein the one or more agents are one or more antibody or antibody fragment capable of binding the one or more cell surface receptors.

Clause 24. The method of clause 22, wherein the one or more agents activate one or more T cell subtype.

Clause 25. The method of clause 22, wherein the one or more agents comprise one or more antibody that binds to one or more T cell surface receptor selected from the group consisting of:
(a) CD3,
(b) CD5,
(c) CD6,
(d) CD28,
(e) CD137, and
(f) CD278.

Clause 26. A method for preferentially expanding one or more subsets of T cells present in a mixed population of T cells, wherein the T cells are expanded in the absence of serum and where the T cells are expanded in a culture vessel having a gas permeable membrane.

Clause 27. The method of clause 26, wherein the T cells have a maximum doubling time of from about 25 hours to about 40 hours.

Clause 28. The method of clauses 26 or 27, wherein the T cells are expanded in the presence of one or more chemokine or cytokine.

Clause 29. The method of clause 28, the one or more chemokine or cytokine is one or more protein selected from the group consisting of:
(a) Interleukin-1α,
(b) Interleukin-2,
(c) Interleukin-4,
(d) Interleukin-1β,
(e) Interleukin-6,
(f) Interleukin-12,
(g) Interleukin-15,
(h) Interleukin-18,
(i) Interleukin-21, and
(j) Transforming growth factor β1.

Clause 30. The method of clauses 26 to 29, wherein one or more of the T cell subsets preferentially expands over one or more different T cell subsets.

Clause 31. The method of clauses 26 to 30, wherein memory T cells preferential expand over antigen specific T cells.

Clause 32. The method of clause 31, wherein memory T cells expand at a rate that is from 5 to 15 times faster than antigen specific T cells.

Clause 33. The method of clauses 26 to 32, wherein the total T cell population expands at a rate that is from 5 to 15 times faster than antigen specific T cells.

Clause 34. The method of clauses 26 to 33, wherein the regulatory T cells expand at a rate that is from 5 to 15 times faster than antigen specific T cells.

Clause 35. The method of clauses 26 to 34, wherein the T cells are cultured in a G-REX® culture vessel.

Clause 36. The method of clause 35, wherein the G-REX® culture vessel is G-REX® 6M Well Plate (Wolf Wilson Corporation, part number 80660M).

Clause 37. The method of clauses 26 to 36, wherein the T cells are cultured in the presence of serum albumin

Clause 38. The methods of clauses 26 to 36, wherein T cells are cultured in a culture medium comprising OPTMIZER™ CTS™ SFM (Thermo Fisher Scientific, cat. no. A1048501).

Clause 39. The methods of clauses 26 to 36, wherein T cells are cultured in a culture medium comprising CTS™ Immune Cell Serum Replacement (ICSR) (Thermo Fisher Scientific, catalog number A2596101).

Clause 40. The methods of clause 26 clauses 26 to 36, wherein T cells are cultured in a culture medium comprising OPTMIZER™ CTS™ SFM (Thermo Fisher Scientific, cat. no. A1048501) and CTS™ Immune Cell Serum Replacement (ICSR) (Thermo Fisher Scientific, catalog number A2596101).

Clause 41. A method for the activation and expansion of T cells, the method comprising:
(a) activating the T cells, and
(b) expanding the T cells,

wherein the T cells are expanded under conditions wherein they have a maximum doubling time of from about 25 hours to about 40 hours, and

wherein the T cells are expanded in the absence of serum.

Clause 42. The method of clause 41, wherein the T cells are purified prior to activation.

Clause 43. The method of clause 42, wherein the T cells are purified by negative selection or positive selection.

Clause 44. The method of clause 43, wherein the negative selection or positive selection occur by either removing or collecting T cells by the use of one or more agents that bind to CD2 receptors or CD3 receptors.

Clause 45. The method of clause 44, wherein the one or more agents that bind to CD2 receptors or CD3 receptors are anti-CD2 and anti-CD3 antibodies.

Clause 46. A method for expanding cells of a T cell subset, the method comprising:
(a) purifying members of a T cell subset, and
(b) culturing the members of the T cell subset obtained in (a),

wherein the T cells are expanded under conditions wherein they have a maximum doubling time of from about 25 hours to about 40 hours, and

wherein the T cells are expanded in the absence of serum.

Clause 47. The method of clause 46, wherein the T cell subset is selected from the groups consisting of:
(a) Th1 T cells,
(b) Th2 T cells,
(c) Th17 T cells,
(d) Th22 T cells,
(e) regulatory T cells,
(f) naïve T cells,
(g) antigen specific T cells,
(h) central memory T cells,
(i) effector memory T cells,
(j) tissue resident memory T cells, and
(k) virtual memory T cells.

Clause 48. The method of clauses 46 to 47, wherein the members of the T cell subset are purified by (1) selective expansion and/or (2) positive of negative selection.

Clause 49. The method of clause 48, wherein the negative selection or positive selection occur by either removing or collecting T cells by the use of one or more agents that bind to one or more cell surface markers.

Clause 50. The method of clause 49, wherein the one or more cell surface marker is a surface marker selected from the group consisting of:
(a) CD2 receptors,
(b) CD3 receptors,
(c) CD8 receptors,
(d) CD19 receptors,
(e) CD20 receptors, and
(f) CD28 receptors.

Clause 51. The method of clauses 49 to 50, wherein the one or more agents that bind to the one or more surface markers are anti-surface marker antibodies.

Clause 52. A method for generating a population of activated, engineered T cells, the method comprising:
(a) introducing into the population of T cells a nucleic acid molecule that encodes protein under conditions where the protein is expressed in the T cells, wherein the protein is a cell surface protein, to produce a population of engineered T cells,
(b) activating members of the population of engineered T cells, and
(c) expanding activating members of the population of engineered T cells to produce the population of activated, engineered T cells,

wherein the T cells are expanded under conditions wherein they have a maximum doubling time of from about 25 hours to about 40 hours, and

wherein the T cells are expanded in the absence of serum.

Clause 53. The method of clause 52, further comprising purifying a T cell subset prior to introducing into the population of T cells the nucleic acid molecule that encodes protein.

Clause 54. The method of clause 53, wherein the protein is a fusion protein.

Clause 55. The method of clause 54, wherein the fusion protein is a chimeric antigen receptor.

Clause 56. The method of clauses 52 to 55, wherein the population of engineered T cells are expanded in the presence of at least one cytokine.

Clause 57. The method of clause 56, wherein the cytokine is Intereukin-2.

Clause 58. The method of clause 52 to 56, wherein the population of engineered T cells are expanded in the presence of an L-alanyl-L-glutamine dipeptide.

Clause 59. The method of clause 58, wherein the L-alanyl-L-glutamine dipeptide is present at a concentration of between from about 1 mM to about 20 mM.

What is claimed is:

1. A method for culturing T cells, the method comprising culturing the T cells under conditions where the T cells have a peak population maximum doubling time of from about 25 hours to about 40 hours, and wherein the T cells are cultured without serum.

2. The method of claim 1, wherein the T cells are cultured in the presence of serum albumin.

3. The methods of claim 2, wherein the serum albumin is human serum albumin.

4. The methods of claim 2, wherein the serum albumin is recombinant serum albumin.

5. The methods of claim 4, wherein the recombinant serum albumin is produced in a cell which is not a mammalian cell.

6. The methods of claim 1, wherein T cells are cultured in a culture medium comprising OPTMIZER™ CTS™ SFM (Thermo Fisher Scientific, cat. no. A1048501).

7. The methods of claim 1, wherein T cells are cultured in a culture medium comprising CTS™ Immune Cell Serum Replacement (ICSR) (Thermo Fisher Scientific, catalog number A2596101).

8. The methods of claim 1, wherein T cells are cultured in a culture medium comprising OPTMIZER™ CTS™ SFM (Thermo Fisher Scientific, cat. no. A1048501) and CTS™ Immune Cell Serum Replacement (ICSR) (Thermo Fisher Scientific, catalog number A2596101).

9. The method of claim 1, wherein T cells are cultured in an incubator where the O₂ concentration is between 15% and 25%.

10. The method of claim 1, wherein T cells are cultured in an incubator where the CO₂ concentration is between 3% and 7%.

11. The method of claim 1, wherein T cells are cultured at a temperature between 34° C. and 40° C.

12. The method of claim 1, wherein T cells are cultured in the presence of a gas permeable membrane.

13. The method of claim 12, wherein the T cells are cultured in a G-REX® culture vessel.

14. The method of claim 13, wherein the G-REX® culture vessel is G-REX® 6M Well Plate (Wolf Wilson Corporation, part number 80660M).

15. The method of claim 12, wherein the gas permeable membrane comprises silicone and is 0.005 to 0.007 inches thick.

16. The method of claim 1, wherein the T cells are cultured in the presence of a glutamine source that will not form substantial amounts of ammonia.

17. The method of claim 16, wherein the glutamine source is an L-alanyl-L-glutamine dipeptide.

18. The method of claim 1, wherein the T cells are present in a mixed population of different T cell subtypes.

19. The method of claim 1, wherein the T cells reach a population density of between 1.0×10⁷ and 8.0×10⁷ cells per cm².

20. The method of claim 1, wherein the T cells are obtained from a sample provided by a donor.

21. The method of claim 20, wherein the donor is a human donor.

22. The method of claim 1, wherein the T cells are contacted with one or more agents that bind to one or more cell receptors present on the T cells.

23. The method of claim 22, wherein the one or more agents are one or more antibody or antibody fragment capable of binding the one or more cell surface receptors.

24. The method of claim 22, wherein the one or more agents activate one or more T cell subtype.

25. The method of claim 22, wherein the one or more agents comprise one or more antibody that binds to one or more T cell surface receptor selected from the group consisting of:
(g) CD3,
(h) CD5,
(i) CD6,
(j) CD28,
(k) CD137, and
(l) CD278.

26. A method for preferentially expanding one or more subsets of T cells present in a mixed population of T cells, wherein the T cells are expanded in the absence of serum and where the T cells are expanded in a culture vessel having a gas permeable membrane.

27. The method of claim 26, wherein the T cells have a maximum doubling time of from about 25 hours to about 40 hours.

28. The method of claim 26, wherein the T cells are expanded in the presence of one or more chemokine or cytokine.

29. The method of claim 29, the one or more chemokine or cytokine is one or more protein selected from the group consisting of:
(k) Interleukin-1α,
(l) Interleukin-2,
(m) Interleukin-4,
(n) Interleukin-1β,
(o) Interleukin-6,
(p) Interleukin-12,
(q) Interleukin-15,
(r) Interleukin-18,
(s) Interleukin-21, and
(t) Transforming growth factor β1.

30. The method of claim 26, wherein one or more of the T cell subsets preferentially expands over one or more different T cell subsets.

31. The method of claim 26, wherein memory T cells preferential expand over antigen specific T cells.

32. The method of claim 31, wherein memory T cells expand at a rate that is from 5 to 15 times faster than antigen specific T cells.

33. The method of claim 26, wherein the total T cell population expands at a rate that is from 5 to 15 times faster than antigen specific T cells.

34. The method of claim 26, wherein the regulatory T cells expand at a rate that is from 5 to 15 times faster than antigen specific T cells.

35. The method of claim 26, wherein the T cells are cultured in a G-REX® culture vessel.

36. The method of claim 35, wherein the G-REX® culture vessel is G-REX® 6M Well Plate (Wolf Wilson Corporation, part number 80660M).

37. The method of claim 26, wherein the T cells are cultured in the presence of serum albumin.

38. The methods of claim 26, wherein T cells are cultured in a culture medium comprising OPTMIZER™ CTS™ SFM (Thermo Fisher Scientific, cat. no. A1048501).

39. The methods of claim 26, wherein T cells are cultured in a culture medium comprising CTS™ Immune Cell Serum Replacement (ICSR) (Thermo Fisher Scientific, catalog number A2596101).

40. The methods of claim 26, wherein T cells are cultured in a culture medium comprising OPTMIZER™ CTS™ SFM (Thermo Fisher Scientific, cat. no. A1048501) and CTS™ Immune Cell Serum Replacement (ICSR) (Thermo Fisher Scientific, catalog number A2596101).

41. A method for the activation and expansion of T cells, the method comprising:
(c) activating the T cells, and
(d) expanding the T cells,
wherein the T cells are expanded under conditions wherein they have a maximum doubling time of from about 25 hours to about 40 hours, and
wherein the T cells are expanded in the absence of serum.

42. The method of claim 41, wherein the T cells are purified prior to activation.

43. The method of claim 42, wherein the T cells are purified by negative selection or positive selection.

44. The method of claim 43, wherein the negative selection or positive selection occur by either removing or collecting T cells by the use of one or more agents that bind to CD2 receptors or CD3 receptors.

45. The method of claim 44, wherein the one or more agents that bind to CD2 receptors or CD3 receptors are anti-CD2 and anti-CD3 antibodies.

46. A method for expanding cells of a T cell subset, the method comprising:
(c) purifying members of a T cell subset, and
(d) culturing the members of the T cell subset obtained in (a),
wherein the T cells are expanded under conditions wherein they have a maximum doubling time of from about 25 hours to about 40 hours, and
wherein the T cells are expanded in the absence of serum.

47. The method of claim 46, wherein the T cell subset is selected from the groups consisting of:
(l) Th1 T cells,
(m) Th2 T cells,
(n) Th17 T cells,
(o) Th22 T cells,
(p) regulatory T cells,
(q) naïve T cells,
(r) antigen specific T cells,
(s) central memory T cells,
(t) effector memory T cells,
(u) tissue resident memory T cells, and
(v) virtual memory T cells.

48. The method of claim 46, wherein the members of the T cell subset are purified by (1) selective expansion and/or (2) positive of negative selection.

49. The method of claim 48, wherein the negative selection or positive selection occur by either removing or collecting T cells by the use of one or more agents that bind to one or more cell surface markers.

50. The method of claim 49, wherein the one or more cell surface marker is a surface marker selected from the group consisting of:
(g) CD2 receptors,
(h) CD3 receptors,
(i) CD8 receptors,
(j) CD19 receptors,
(k) CD20 receptors, and
(l) CD28 receptors.

51. The method of claim 49, wherein the one or more agents that bind to the one or more surface markers are anti-surface marker antibodies.

52. A method for generating a population of activated, engineered T cells, the method comprising:
(d) introducing into the population of T cells a nucleic acid molecule that encodes protein under conditions where the protein is expressed in the T cells, wherein the protein is a cell surface protein, to produce a population of engineered T cells,
(e) activating members of the population of engineered T cells, and
(f) expanding activating members of the population of engineered T cells to produce the population of activated, engineered T cells,
wherein the T cells are expanded under conditions wherein they have a maximum doubling time of from about 25 hours to about 40 hours, and
wherein the T cells are expanded in the absence of serum.

53. The method of claim 52, further comprising purifying a T cell subset prior to introducing into the population of T cells the nucleic acid molecule that encodes protein.

54. The method of claim 53, wherein the protein is a fusion protein.

55. The method of claim 54, wherein the fusion protein is a chimeric antigen receptor.

56. The method of claim 52, wherein the population of engineered T cells are expanded in the presence of at least one cytokine.

57. The method of claim 56, wherein the cytokine is Intereukin-2.

58. The method of claim 52, wherein the population of engineered T cells are expanded in the presence of an L-alanyl-L-glutamine dipeptide.

59. The method of claim 58, wherein the L-alanyl-L-glutamine dipeptide is present at a concentration of between from about 1 mM to about 20 mM.
